(19) United States
(12) Patent Application Publication
(10) Pub. No.: US 2019/0064147 A1
(43) Pub. Date: Feb. 28, 2019

(54) FORMULATIONS AND SCREENING OF BIOLOGICAL THERAPEUTIC AGENTS

(71) Applicant: Biogen MA Inc., Cambridge, MA (US)

(72) Inventors: Mark Krebs, Leiden (NL); Mariana Dimitrova, Medford, MA (US); Kapil Gupta, Redmond, WA (US); Shantanu Sule, Arlington, MA (US); Randall Mauldin, Bedford, MA (US); Adnan Zunic, Cambridge, MA (US); Lori Karpes, Quincy, MA (US)

(73) Assignee: Biogen MA Inc., Cambridge, MA (US)

(21) Appl. No.: 15/544,315

(22) PCT Filed: Jan. 18, 2016
(86) PCT No.: PCT/US2016/013810

§ 371 Date: Jul. 18, 2017

Related U.S. Application Data
(60) Provisional application No. 62/104,798, filed on Jan. 18, 2015.

(51) Int. Cl. G01N 33/50; A61K 47/12; G01N 33/68; A61K 47/18; A61K 47/26; G01N 33/84; G01N 33/94; C07K 1/113; G01N 33/15

(52) U.S. Cl. CPC G01N 33/5008; A61K 47/12; G01N 33/6803; A61K 47/183; G01N 2500/04; G01N 33/84; G01N 33/94; C07K 1/1136; G01N 33/15; A61K 47/26

(57) ABSTRACT

Provided herein, in some aspects, are methods of developing a biological therapeutic product. The methods can be used to formulate a biological therapeutic agent or screen biological therapeutic agents.

### RELATED APPLICATIONS

This application is a national stage filing under 35 U.S.C. § 371 of PCT International Application PCT/US2016/013810 filed Jan. 18, 2016 entitled “FORMULATIONS AND SCREENING OF BIOLOGICAL THERAPEUTIC AGENTS,” which claims the benefit under 35 U.S.C. § 119(e) of U.S. provisional application No. 62/104,798, filed Jan. 18, 2015, the contents of each of which are incorporated by reference herein in their entirety.

### FIELD OF THE INVENTION

The present disclosure relates, in some aspects, to methods of developing a biological therapeutic product.

### BACKGROUND OF INVENTION

Development of biological therapeutic drug products is a long and challenging process. In particular, formulations may require more resource and effort than conventional small molecule pharmaceuticals due to the complex components involved. Formulation is one of the critical steps in developing a biological candidate as a therapeutic product. Development of stable biological therapeutic formulations may require more resource and effort than conventional small molecule pharmaceuticals due to the complex components involved. For example, proteins typically have more stability issues as a result of their delicate structural stability. A major challenge is to maintain the integrity of the biological therapeutic drug products during routine pharmaceutical processing, storage, handling, and delivery to the patient. Because biological therapeutic drug products are complex molecules composed of numerous reactive chemical groups and delicate three-dimensional structures, it is difficult to identify all formulation variables and appropriate combinations thereof to avoid aggregation or degradation.

### SUMMARY OF INVENTION

Aspects of the disclosure relate to methods and compositions for evaluating the properties of biological products (e.g., of therapeutic or candidate protein products) in order to assess their biochemical and/or physical stabilities and/or their solution properties. In some embodiments, aspects of the disclosure provide systematic techniques for evaluating the properties of a protein product under different pH conditions, in response to different buffers, and/or in the presence of different excipients.

Aspects of the disclosure are useful to screen protein drug candidates in order to identify ones that are more stable for liquid formulation purposes. Aspects of the disclosure also provide systematic methods for identifying liquid formulations that are suitable for maintaining or promoting the stability of one or more biological protein products. In some embodiments, methods described herein can be used to accelerate the development of protein formulations. In some embodiments, methods described herein can be useful to screen protein compositions and identify formulation limitations using small amounts of protein product (which is often expensive to produce in large quantities) by systematically evaluating the effects of different composition elements (e.g., pH, buffer, and/or excipients) independently without performing large scale screens that include multiple combinations of different composition elements. In some embodiments, methods described herein are useful to identify formulations that support high concentrations of protein products. In some embodiments, high concentration therapeutic protein formulations can be used for low volume administration to patients, for example for low volume subcutaneous dosing of a therapeutic protein.

The present disclosure provides, in some aspects, methods of developing a biological therapeutic product (e.g., a protein therapeutic or a candidate protein product). The provided methods can be used to formulate at least one biological therapeutic product. In other embodiments, the provided methods can be used to screen at least one biological therapeutic product. The provided methods take a systematic approach to evaluate one or more properties or a biological product (e.g., physical, biochemical, and/or solution properties) under various conditions such as different pH conditions, buffers, and excipients. In some embodiments, the effect of pH on the one or more properties of a biological product is used to identify an acceptable pH range for subsequent buffer evaluation. In turn, in some embodiments the effect of different buffers on one or more properties of a biological product can lead to the selection of one or more acceptable buffers for excipient evaluation. In some embodiments, the effect of different excipients on one or more properties of a biological product in the presence of a selected buffer within an identified pH range can provide an acceptable formulation that supports stability of the biological product. In some embodiments, a formulation that is suitable for high concentration delivery of the biological product can be developed as described herein.

In some aspects, one or more pH, buffer, and/or excipient conditions (e.g., ranges and/or concentrations) that are used are selected to stress the stability and/or solubility of the candidate therapeutic agent. In some embodiments, one or more additional conditions are used to stress a candidate therapeutic agent (e.g., to accelerate the effects of pH, buffer, and/or excipient). In some embodiments, one or more additional stresses can include high temperature (e.g., above room temperature, above 30° C., above 40° C., etc.), freeze-thawing, agitating, or other physical stresses. By stressing a candidate therapeutic agent (e.g., a protein), in some embodiments, information can be obtained that can be used to a) identify conditions (e.g., pH, buffer, and/or excipients) that are useful to formulate a candidate therapeutic agent so as to promote stability and/or solubility of the agent, and/or b) identify one or more susceptibilities of a candidate therapeutic agent to physico-chemical and/or solution changes that may not be desirable for a pharmaceutical formulation. In some embodiments, this information can be used to help formulate a therapeutic agent (e.g., a protein) to protect the agent from unwanted physico-chemical and/or solution changes.

In other aspects, two or more candidate therapeutic agents can be prioritized or compared based on their performance under different conditions as described herein.

In some embodiments, the provided methods involve a systematic evaluation of pH, buffer, and excipient effect and are more efficient and effective than traditional blind testing for the development and comparison of biologic product formulations. In some embodiments, a range of pH conditions are evaluated first. In some embodiments, the effects of one or more different buffers are then evaluated (e.g., within a pH range that was identified as being acceptable). In some embodiments, the effects of one or more excipients are then evaluated (e.g., in a buffer that was identified as acceptable).

In some embodiments, the provided methods can be used to develop time- and protein-efficient assays allowing for the determination of likely degradation pathways and differentiation and ranking of biological therapeutic agents in a research and development program prior to transition to formulation. In some embodiments, the provided methods can be used to identify product characteristics that are either amenable to or inappropriate for further development and manufacturing.

In some aspects, the present disclosure also provides methods for developing pharmaceutical formulations with a viscosity that is suitable for clinical use. In some embodiments, the formulation prepared from the provided method has a viscosity suitable for injection, e.g., through a needle or other suitable device.

In some embodiments, a method of assisting in the selection of a candidate therapeutic agent (e.g., protein) for pharmaceutical formulation comprises a) evaluating a pH effect on one or more properties of a plurality of candidate therapeutic proteins; b) evaluating a buffer effect on one or more properties of the plurality of candidate therapeutic proteins; c) evaluating an excipient effect on one or more properties of the plurality of candidate therapeutic proteins; and, determining a relative stability of each of the plurality of candidate therapeutic proteins based on the pH, buffer, and excipient effects of a) through c), wherein candidate therapeutic proteins that have relatively higher stability are more suitable for pharmaceutical formulation.

In some embodiments, a method of selecting a pharmaceutical formulation for a candidate therapeutic agent (e.g., protein) comprises a) evaluating a pH effect on one or more properties of a candidate therapeutic protein and determining a pH range within which the candidate therapeutic protein is relatively stable and/or soluble; b) evaluating a buffer effect on the candidate therapeutic protein within the pH range of a) and determining a relative stability and/or solubility of the candidate therapeutic protein in two or more different buffers; c) evaluating an excipient effect on one or more properties of the candidate therapeutic protein within the pH range of a) and in a buffer of b) in which the candidate therapeutic protein is relatively stable and/or soluble; and, determining a formulation for the candidate therapeutic protein that is within the pH range of a), comprises the buffer of b), and comprises the excipient of c).

In some embodiments, a method of selecting a pharmaceutical formulation for a therapeutic agent (e.g., a protein) at high concentration comprises a) evaluating a pH effect on one or more properties of a candidate therapeutic protein and determining a pH range within which the candidate therapeutic protein is relatively stable and/or soluble; b) evaluating a buffer effect on the candidate therapeutic protein within the pH range of a) and determining a relative stability and/or solubility of the candidate therapeutic protein in two or more different buffers; c) evaluating an excipient effect on one or more properties of the candidate therapeutic protein within the pH range of a) and in a buffer of b) in which the candidate therapeutic protein is relatively stable and/or soluble; and, determining a formulation for the candidate therapeutic protein that is within the pH range of a), comprises the buffer of b), and comprises the excipient of c).

In some embodiments, the stability of a candidate therapeutic agent (e.g., protein) is evaluated by determining conformational stability, charge, charge isoforms, integrity, and/or chemical modification of the candidate therapeutic agent (e.g., protein) under conditions a), b), and/or c) above. In some embodiments, the solubility of a candidate therapeutic agent (e.g., protein) is evaluated by determining aggregation of the candidate therapeutic protein under conditions a), b), and/or c) above.

In some embodiments, a candidate therapeutic agent (e.g., protein) is selected by comparing stability and/or solubility of the candidate therapeutic agent to a reference agent (e.g., protein) and/or one or more alternative candidate therapeutic agents (e.g., proteins).

In some embodiments, a formulation is selected by comparing agent (e.g., protein) stability and/or solubility in a formulation to a) a reference agent (e.g., protein) stability and/or solubility and/or b) agent (e.g., protein) stability and/or solubility in one or more alternative formulations.

In some embodiments, the pH effect is evaluated by determining one or more stability and/or solubility properties of a candidate therapeutic agent (e.g., protein) under a plurality of pH conditions (e.g., between pH 3.0 and 9.0, between pH 4.0 and pH 8.0, or within any other suitable pH range described in this document), for example, using one or more known assays and/or assays described in this document.

In some embodiments, the buffer effect is evaluated by determining one or more stability and/or solubility properties of a candidate therapeutic agent (e.g., protein) in a plurality of different buffers, for example, using one or more known assays and/or assays described in this document.

In some embodiments, the excipient effect is evaluated by determining one or more stability and/or solubility properties of a candidate therapeutic agent (e.g., protein) in the presence of a plurality of different excipients, for example, using one or more known assays and/or assays described in this document.

In some embodiments, information from the pH, buffer, and/or excipient studies are used to help select a therapeutic agent (e.g., a protein) for pharmaceutical formulation, and/or to select or develop a formulation for a therapeutic agent of interest. For example, information from the pH screen can be useful to determine how a molecule is likely to fail (e.g., be modified, aggregate, or otherwise undergo unwanted changes) under more extreme conditions. In some embodiments, the excipient study provides information about the behavior of a candidate therapeutic agent under conditions that may be similar to a pharmaceutical formulation (for example, similar pH, buffer, and excipient conditions). In some embodiments, the excipient study can be useful to evaluate the behavior of a candidate agent under high concentrations (e.g., under pH, buffer, and excipient conditions selected as described in this document).

In some embodiments, a method of developing a biological therapeutic product comprises (a) evaluating a pH effect on one or more properties of at least one biological therapeutic agent and identifying an acceptable pH range within which the at least one biological therapeutic agent is stable; (b) evaluating the effect of two or more buffers on one or more properties of the at least one biological therapeutic agent within the acceptable pH range and selecting an acceptable buffer within which the at least one biological therapeutic agent is stable; and (c) evaluating the effect of two or more excipients on one or more properties of the at least one biological therapeutic agent within the acceptable pH range in the acceptable buffer and selecting one or more excipients upon which the at least one biological therapeutic agent is stable.

In some embodiments, the evaluation of the pH effect in step (a) comprises preparing a first batch of multiple solutions each comprising the at least one biological therapeutic agent at a pH from about 1.0 to 13.0 and initially evaluating the one or more properties; incubating the first batch solutions for a first period of time and first evaluating the one or more properties; and optionally further incubating the incubated first batch solutions for a second period of time and evaluating the one or more properties.

In some embodiments, the evaluation of the buffer effect in step (b) comprises preparing a second batch of multiple solutions with two or more buffers, wherein each solution comprises the biological therapeutic agent and a buffer at a pH within the acceptable pH range; initially evaluating the one or more properties of the biological therapeutic agent in each solution; incubating the solutions of the second batch for a third period of time and evaluating the one or more properties of the biological therapeutic agent in each solution; and further optionally incubating the solutions of the second batch for a fourth period of time and evaluating the one or more properties of the biological therapeutic agent in each solution.

In some embodiments, the evaluation of the excipient effect in step (c) comprises preparing a third batch of multiple solutions each comprising the biological therapeutic agent, the optimal buffer, and one or more excipients at the acceptable pH; initially evaluating the one or more properties of the biological therapeutic agent in each solution; incubating the solutions of the third batch for a fifth period of time and evaluating the one or more properties of the biological therapeutic agent in each solution; and optionally further incubating the solutions for a sixth period of time and evaluating the one or more properties of the biological therapeutic agent in each solution.

In some embodiments, one or more preparations (e.g., protein preparations), for example the solutions of the third batch described above, are subjected to agitation stress (e.g., for a fifth period of time) and one or more properties are evaluated. In some embodiments, the one or more preparations (e.g., protein preparations) are subjected to agitation stress (e.g., for a sixth period of time) after a prior agitation stress, and one or more properties are evaluated.

In some embodiments, one or more preparations (e.g., protein preparations), for example the solutions of the third batch described above, are subjected to freeze-thaw stress (e.g., for a fifth period of time) and one or more properties are evaluated. In some embodiments, the one or more preparations (e.g., protein preparations) are subjected to agitation stress (e.g., for a sixth period of time) after the freeze-thaw stress, and one or more properties are evaluated.

In some embodiments, one or more aggregation and/or degradation properties of one or more biological agents are evaluated (e.g., at one or more time points). In some embodiments, one or more degradation properties are conformation stability, charge stability, and/or integrity (e.g., physical integrity as evaluated by the presence or absence of clipping or other degradation products). In some embodiments, one or more properties are measured by differential scanning calorimetry (DSC), size exclusion chromatography (SEC), gel electrophoresis (e.g., using GXII), and/or charge variant analysis (icIEF).

In some embodiments, the one or more excipients are Generally Recognized as Safe (GRAS) excipients. In some embodiments, the one or more excipients are selected from the group consisting of sugars, polyols, amino acids, and polymers.

In some embodiments, the concentrations of the biological therapeutic agents (e.g., in the first batch solutions and second batch solutions) are about 1 mg/mL. In some embodiments, the concentrations of the biological therapeutic agent in the third batch solutions are about 50 mg/mL. In some embodiments, the concentrations of biological therapeutic agent in the third batch solutions are about 1 mg/mL when subjecting to agitation stress.

In some embodiments, the pH in step (a) is from about 1.0 to 12.0, e.g., about 2.0 to 11.0, about 3.0 to 10.0, about 4.0 to 9.0, about 4.0 to 8.0, about 5.0 to 9.0, or about 5.0 to 8.0.

In some embodiments, the one or more properties being evaluated are assayed at least once a day, once every few days, or once a week. In some embodiments, the first period of time is about a week. In some embodiments, the second period of time is about a week.

In some embodiments, methods described in this application are useful to formulate, or to assist in the formulation, of the at least one biological therapeutic agent. In some embodiments, methods described in this application are useful to screen, or to assist in the screening of, the at least one biological therapeutic agent (e.g., a plurality of candidate biological therapeutic agents) to identify a biological therapeutic product candidate for formulation and/or further development.

In some embodiments, a biological therapeutic product candidate has one or more acceptable properties in the assays described in this application (e.g., in the second and/or third batch solutions above). In some embodiments, the acceptable properties are acceptable aggregation and degradation properties.

In some embodiments, the at least one biological therapeutic agent is a protein. In some embodiments, the protein is an antibody.

These and other aspects are described in more detail in the following description and are illustrated by the following non-limiting drawings and examples.

### BRIEF DESCRIPTION OF DRAWINGS

FIG. 1 depicts a graph showing the conformational stability of candidate proteins along the pH spectrum measured by Differential Scanning calorimetry (DSC). The candidate proteins are mixed in a citrate buffer at a pH 2, 3, 4, 5, 6, 7, 8, 9, 10, and 11. Candidate protein 3 appears to have a greater risk of conformation issue due to instability in the Fab region.

FIG. 2 depicts a graph showing Size Exclusion Chromatography (SEC) analysis of pH effect on the candidate proteins. The percent (%) of high molecular weight (HMW) species indicates the amount of protein aggregates formed. The data were collected from each formulation after incubation for two weeks at 40° C. Each formulation contains 1 mg/mL candidate protein, 20 mM citrate, and 150 mM arginine (Arg) at a pH of 2, 3, 4, 5, 6, 7, 8, 9, 10, or 11. Lower instability appears to be present at the lower pHs.

FIG. 3 depicts a graph showing the GXII lab chip monomer integrity analysis along the pH spectrum. The percent (%) of low molecular weight (LMW) species indicates presence of protein monomers. The data were collected from each formulation after incubation for two weeks at 40° C. Each formulation contains about 1 mg/mL candidate protein, 20 mM citrate, 150 mM arginine (Arg), and has a pH value of 2, 3, 4, 5, 6, 7, 8, 9, 10, or 11. Lower instability appeared to be present at the lower pHs.

FIG. 4 depicts a graph showing the conformational stability of four candidate proteins at three pH values in three buffer systems. The data were collected by the Differential Scanning calorimetry (DSC). Buffer A is 20 mM phosphate; Buffer B is 20 mM His; Buffer C is 20 mM citrate; pH A is pH 6; pH B is pH 7; and pH C is pH 8. The DSC analysis indicates that Buffer B appears to reduce conformational stability across the candidate proteins. Within the same buffer system, pH has limited impact on conformation retention. Candidate protein 3 continues to exhibit lower Fab conformational stability across the three buffers.

FIG. 5 depicts a graph showing the protein integrity GXII lab chip monomer integrity analysis of the buffer system effects. It was observed that protein integrity has a pH-dependent relationship in all buffers, though less pronounced in Buffer C. This relationship correlates increasing pH with loss of monomer integrity. Candidate protein 2 appears to retain a higher amount of monomer. Buffer A, Buffer B, Buffer C, pH A, pH B, and pH C are as defined in FIG. 4.

FIG. 6 depicts a graph showing the Size Exclusion Chromatography (SEC) analysis of buffer effect. It was observed that protein aggregation has a pH-dependent relationship in all buffers, though less pronounced in Buffer C. This relationship correlates increasing pH with increasing heterogeneity. Candidate protein 3 appears to contain consistently lower aggregate levels. Buffer B appears to offer greater protection against aggregation. Buffer A, Buffer B, Buffer C, pH A, pH B, and pH C are as defined in FIG. 4.

FIG. 7 depicts a graph showing the iCIEF analysis of buffer effect (BioProcess Int. 2011, 9(10), 48-54). It was observed that protein charge isoforms have a pH-dependent relationship in all buffers, though less pronounced in Buffer C. Buffer A, Buffer B, Buffer C, pH A, pH B, and pH C are as defined in FIG. 4.

FIG. 8 depicts a graph showing the Differential Scanning calorimetry (DSC) analysis of candidate proteins in different formulations. These results indicate alteration of conformational stability by the addition of some investigated excipients and formulations. It was observed that Candidate protein 3's Fab region is consistently less stable than that of the other candidates. Conditions: Formulation 1: 0.5 mg/mL candidate protein, 20 mM Citrate, pH 6.5; Formulation 2: 0.5 mg/mL candidate protein, 20 mM Citrate, pH 6.5, 0.05% PS80; Formulation 3: 0.5 mg/mL candidate protein, 20 mM Citrate, pH 6.5, 150 mM Arg; Formulation 4: 0.5 mg/mL candidate protein, 20 mM Citrate, pH 6.5, 150 mM Arg, 0.05% PS80; Formulation 5: 0.5 mg/mL candidate protein, 20 mM Citrate, pH 6.5, 150 mM NaCl; Formulation 6: 0.5 mg/mL candidate protein, 20 mM Citrate, pH 6.5, 150 mM NaCl, 0.05% PS80; Formulation 7: 0.5 mg/mL candidate protein, 20 mM Citrate, pH 6.5, 5% sucrose; Formulation 8: 0.5 mg/mL candidate protein, 20 mM Citrate, pH 6.5, 5% sucrose, 0.05% PS80.

FIG. 9 depicts a graph showing the excipient effect on monomer integrity. The GXII lab chip analysis of protein solutions were held in accelerated conditions for 2 weeks. Formulations 1-8 are as defined in FIG. 8.

FIG. 10A depicts a graph showing the excipient effects on the charge isoforms. The iCIEF analysis of protein solutions was held in accelerated conditions for 2 weeks. FIG. 10B depicts a graph showing the aggregate levels in solutions upon delivery from Research (Table 1). Formulations 1-8 are as defined in FIG. 8.

FIG. 11 depicts a graph showing the aggregation of candidate proteins at high concentrations in various formulations. It was found % HMW increase after 2 weeks at 40° C. Significant aggregation was observed for all four candidates in all basic formulations. It indicates that these candidates are aggregation-prone and it is likely challenging to formulate. Formulations 1-8 are as defined in FIG. 8.

FIG. 12 depicts a graph showing the aggregation of candidate proteins upon agitation. Formulations 1-8 are as defined in FIG. 8.

FIG. 13 depicts a graph showing the aggregation of candidate proteins upon freeze-thaw. Formulations 1-8 are as defined in FIG. 8.

FIGS. 14A-14C depict graphs showing DSC of different candidate proteins. FIG. 14A depicts a graph showing the DSC unfolding curves of Candidate A at the indicated pH levels.

FIG. 14B depicts a graph showing the DSC unfolding curves of Candidate B at the indicated pH levels. FIG. 14C depicts a graph showing the DSC unfolding curves of Candidate C at the indicated pH levels.

FIGS. 15A-15C depict graphs showing an analysis of pH effect on Candidate A (FIG. 15A), Candidate B (FIG. 15B), and Candidate C (FIG. 15C) aggregation. The total high molecular weight (HMW) species indicates the amount of protein aggregates formed.

FIGS. 16A-16C depict graphs showing an analysis of pH effect on the formation of LMW species in Candidate A (FIG. 16A), Candidate B (FIG. 16B), and Candidate C (FIG. 16C).

FIGS. 17A-17C depict graphs showing a non-reduced intact GXII lab chip monomer integrity analysis along the pH spectrum in Candidate A (FIG. 17A), Candidate B (FIG. 17B), and Candidate C (FIG. 17C).

FIGS. 18A-18C depict graphs showing the percent acidic species formation along the pH spectrum in Candidate A (FIG. 18A), Candidate B (FIG. 18B), and Candidate C (FIG. 18C).

FIGS. 19A-19C depict graphs showing imaging Capillary IsoElectric Focusing (iCIEF) analysis of Candidate A (FIG. 19A), Candidate B (FIG. 19B), and Candidate C (FIG. 19C) in the indicated buffers.

FIGS. 20A-20C depict graphs regarding the solubility of the candidate proteins. FIG. 20A shows the normalized peak area of Candidate B formulated in citrate buffer over increasing percentages of PEG. FIG. 20B shows the theoretical maximal concentration of each candidate protein formulated in the two indicated buffers. FIG. 20C shows the midpoint of the percent PEG of each candidate protein formulated in the two indicated buffers.

FIGS. 21A-21C depict graphs showing SEC analysis of buffer effect in the three candidate proteins. In FIG. 21A, the upper graph shows the Candidate A monomer, the middle graph shows the Candidate A HMW, and the lower graph shows the Candidate A LMW. In FIG. 21B, the upper graph shows the Candidate B monomer, the middle graph shows the Candidate B HMW, and the lower graph shows the Candidate B LMW. In FIG. 21C, the upper graph shows the Candidate C monomer, the middle graph shows the Candidate C HMW, and the lower graph shows the Candidate C LMW.

FIGS. 22A-22B depict the interaction parameter (k_{D}) as determined by dynamic light scattering (DLS) in three candidate proteins. The results are represented graphically in FIG. 22A and tabulated in FIG. 22B.

FIGS. 23A-23B depict graphs showing the aggregation of candidate proteins upon agitation (FIG. 23A) and freeze-thaw (FIG. 23B).

FIGS. 24A-24B depict biophysical characterizations of three candidate proteins. FIG. 24A shows the hydrophobicity of the candidates as determined through analytical hydrophobic interaction chromatography (HIC). FIG. 24B shows the thermal stability of the three candidate proteins.

FIGS. 25A-25B depict graphs showing the percent change in acid species in Candidate A (FIG. 24A) and Candidate C (FIG. 24B) in the indicated buffers.

FIGS. 26A-26C depict high concentration excipient screening (100 mg/mL). The candidates were formulated in histidine buffer, pH 6, in combination with several common excipients, with or without 0.05% polysorbate 80. The change in percent HMW species using the best formulation is given in FIG. 26A. FIG. 26B shows the change in percent acid species using the best formulation. FIG. 26C tabulates the results from FIGS. 26A-26B.

FIGS. 27A-27B depict the results of a solubility assessment using PEG precipitation. The results of the assays of Candidate A (FIG. 27A) and Candidate C (FIG. 27B) in the indicated formulations at pH 6 are shown.

FIG. 28 shows the low pH hold and control. Candidate C was held at a low pH for 5 hours prior to testing and compared to a control.

### DETAILED DESCRIPTION OF INVENTION

Aspects of the disclosure relate to systematic approaches for evaluating formulation properties (e.g., liquid formulation properties) of one or more biological products. In some embodiments, properties (e.g., biochemical and/or physical stabilities and/or solution properties) of a biological product in solution are evaluated over a broad pH range, for example in a range of pH 1-12 (e.g., from around pH 2 or pH 3 to around pH 10 or pH 11, for example from around pH 4 or pH 5 to around pH 8 or pH 9). In some embodiments, the effects of different buffer systems are evaluated on the properties of one or more biological products in solution. In some embodiments, the buffer systems that are evaluated are selected based on the information obtained from the pH studies. For example, in some embodiments buffers that are evaluated are ones that have good buffering capacity within a pH range that was identified as being suitable for formulation. In some embodiments, the effects of one or more different excipients are evaluated on the properties of one or more biological products in solution. In some embodiments, the effects of one or more excipients are evaluated at a pH and in a buffer system that were selected as being suitable for formulation. It should be appreciated that pH ranges, buffer systems, and excipients can be selected as suitable for formulation based on their effect on biochemical and/or physical stabilities and/or solution properties of a biological product. Conditions that don't promote or that prevent instability or solubility problems can be suitable for formulation. In some embodiments, conditions are selected to allow for high concentrations of a protein product (e.g., greater than 50 mg/mL, greater than 100 mg/mL, greater than 150 mg/mL, greater than 200 mg/mL, greater than 250 mg/mL, or higher) in a formulation that does not result in protein aggregation or viscosity levels that would prevent delivery to s subject via injection (e.g., subcutaneous injection).

Accordingly, this systematic approach can be useful to i) identify a candidate therapeutic that can be readily formulated (e.g., a candidate that is more stable in a pharmaceutically acceptable formulation than other candidates), ii) identify a formulation for use with one or more therapeutics (e.g., a formulation that stabilizes the one or more therapeutics), iii) identify a formulation that can be used for high concentrations of a protein product (e.g., for small volume subcutaneous injection, for example between 0.5 mL and 2 mL, for example around 1 mL injection volumes). It also should be appreciated that methods and compositions described herein can be used to understand the biochemical and physical properties of a biological product and identify behaviors of the product (e.g., tendencies to degrade, aggregate, produce isomers such as ionic or other isomers) under different conditions. In some embodiments, behaviors of a product that are identified under non-optimal conditions (e.g., in the context of pH, buffer, and/or excipient conditions that promote instability) are also observed over time in a product that is formulated under other conditions. Accordingly, product behaviors and/or properties that are identified under non-optimal conditions also can be used to assess and/or monitor the condition of a biological product that is provided in an acceptable formulation (e.g., a formulation that is considered stable and that is pharmaceutically acceptable). Accordingly, one or more behaviors or properties can be monitored when studying or comparing the effectiveness of different formulations. In some embodiments, information about behaviors or properties of one or more biological products can be used to evaluate the stability of a therapeutic preparation. For example, a preparation of therapeutic product can be assayed to determine whether or not the behavior or property is present or to measure the level of the behavior or property in order to determine whether the preparation is suitable for use or whether it has started to degrade. In some embodiments, one or more properties of an agent (e.g., a protein) are evaluated under conditions that stress the physico-chemical and/or solution properties of the agent. Such conditions can include temperature stresses, repeated temperature changes (e.g., freeze-thaw cycles), physical stresses (e.g., agitation or other physical stresses), and/or other stresses that can be used to accelerate the appearance or progression of one or more modifications or other changes in the agent that may not be desirable for a pharmaceutical formulation.

Aspects of the disclosure are useful to help select therapeutic protein products and related formulations for administration to a subject. A subject can be a vertebrate, mammal, including but not limited to, a human, a non-human primate, a rodent, an ovine, a bovine, or other mammal. A subject can be a subject in need of treatment with the therapeutic protein product. It should be appreciated that in some embodiments, formulations described herein can be used for small volume administration (e.g., between 0.5 mL and 2 mL, for example around 1 mL injection volumes). It also should be appreciated that in some embodiments, one or more formulations described herein can be sterilized using any suitable technique (e.g., filtration or other technique) in order to produce a composition that is suitable (e.g., sterile) for administration to a subject.

It should be appreciated that in some embodiments compositions and formulations described herein can be administered by any suitable route, including but not limited to one of: intravenous injection or infusion (IV), subcutaneous injection (SC), intraperitoneally (IP), or intramuscular injection. It is also possible to use intra-articular delivery. Other modes of parenteral administration can also be used. Examples of such modes include: intraarterial, intrathecal, intracapsular, intraorbital, intracardiac, intradermal, transtracheal, subcuticular, intraarticular, subcapsular, subarachnoid, intraspinal, and epidural and intrasternal injection.

In some embodiments, biological products refer to protein products, for example protein products that can be used as therapeutic agents. Therapeutic protein products can be purified from biological material (e.g., from natural sources) or prepared using recombinant techniques (for example from a cell culture that expresses a protein from a recombinant gene). In some embodiments, protein products can be modified (e.g., glycosylated). In some embodiments, protein products can be antibodies (e.g., including but not limited to monoclonal antibodies and/or antibodies with mixed isotypes), hormones, cytokines, chemokines, receptors, ligands, enzymes, growth factors, neurotrophic factors, or other protein products whether they have naturally occurring or recombinant or engineered amino acid sequences (e.g., chimeric or humanized proteins). Methods and compositions described herein can be used to assist in the development of formulations for one or more protein products by systematically evaluating their biochemical and/or physical stabilities and/or solution properties as described herein.

In some embodiments, the behavior of a biological product can be assessed by evaluating one or more (e.g., 2, 3, 4, 5, 5-10, or more) properties of biochemical stability, solubility, and/or physical stability.

In some embodiments, biochemical stability can be assessed by evaluating (e.g., measuring or otherwise determining) one or more of the following properties of a biological (e.g., protein) product: fragmentation, deamidation, oxidation, amino acid isomerization (e.g., Asp isomerization), sequence variation, O-glycosylation, and glycation. However, it should be appreciated that one or more additional properties can be evaluated as aspects of the disclosure are not limited in this respect.

In some embodiments, solution properties can be assessed by evaluating (e.g., measuring or otherwise determining) one or more of the following properties of a biological (e.g., protein) product: reversible self-association, solubility, viscosity, and rheology. However, it should be appreciated that one or more additional properties can be evaluated as aspects of the disclosure are not limited in this respect.

In some embodiments, physical stability can be assessed by evaluating (e.g., measuring or otherwise determining) one or more of the following properties of a biological (e.g., protein) product: conformational stability, colloidal stability, interfacial stability, aggregation, and charge (e.g., isoelectric point—pI, charge isoform formation, and/or other charge related properties). However, it should be appreciated that one or more additional properties can be evaluated as aspects of the disclosure are not limited in this respect.

In some embodiments, the effects of pH on a formulation are evaluated using a selected buffer system that can be prepared to cover a broad range of pH values. This allows the effect of different pH values to be evaluated independently of the effect of different buffers. For example, in some embodiments citrate based buffers can be prepared for different pH values or ranges by using different combinations of citric acid, mono, di, and/or tribasic citrate (for example mono, di, and/or trisodium citrate), even though the buffering capacity of citrate buffers at different pHs may not be the same. It should be appreciated that other buffering systems can be used to evaluated the pH effect as aspects of the disclosure are not limited in this respect. However, in some embodiments the same buffer system (based on the same buffer molecule) is used to prepare buffers at different pHs being tested (as exemplified by the citrate buffer system described above).

It should be appreciated that any suitable techniques can be used to evaluate one or more properties described herein. For example one or more properties can be evaluated using differential scanning calorimetry (DSC), size exclusion chromatography (SEC), reducing and non-reducing gel electrophoresis (e.g., using a GXII Labchip), charge variant analysis (e.g., imaged capillary isoelectric focusing—icIEF). However, it should be appreciated that one or more additional or alternative techniques can be used as aspects of the disclosure are not limited in this respect.

In some embodiments, a hotspot analysis is performed on one or more proteins (e.g., candidate therapeutic proteins). In some embodiments, a sequence analysis is performed to identify one or more amino acid positions that are potential sites for deamidation (e.g., at one or more Asparagine-Glycine or Glutamine-Glycine sequences), oxidation (e.g., at one or more Methionines), isomerization (e.g., at one or more Aspartate-Glycine sequences), glycosylation (e.g., at one or more Asparagine-Serine-Threonine sequences), clipping (e.g., of one or more Aspartate-Proline bonds), and/or other modification). In some embodiments, one or more assays can be performed to determine whether, or the extent to which, one or more of these modifications occurs.

In some embodiments, Differential Scanning calorimetry (DSC) is performed to evaluate one or more proteins. In some embodiments, appropriate sample and buffer volumes for a MicroCal VP-Capillary DSC or TA Instruments Nano DSC are placed in appropriate plates and placed in the instruments. In some embodiments, samples are run at 0.5 mg/mL. In some embodiments, samples are heated from 20 to 110° C. at 90° C./hour. In some embodiments, data are baseline-subtracted and fitted to a non-2 state model to extract Tm-values and ΔH, enthalpies of unfolding. The latter values can be summed to determine the total enthalpy of unfolding. In some embodiments, Tonset, defined as the temperature at which the energy of unfolding is equal to 10% of the maximum for Tm1, is calculated and recorded.

In some embodiments, Size Exclusion Chromatography (SEC) is performed to evaluate one or more proteins. In some embodiments, approximately 10 μg of each sample is injected at each time point onto the SEC equipment (for example, using a Tosoh TSKgel G3000SWxl column, 20 mM Phosphate, 150 mM Sodium Chloride, pH 6.8 running buffer at 1 mL/min). In some embodiments, samples can be analyzed using UPLC-SEC using Waters BEH SEC 200 Å 150 mm column and the same mobile phase as described above. In some embodiments, sample injection can be reduced to approximately 1 μg. In some embodiments, a UPLC-SEC flow rate of 0.4 mL/min can be used. In some embodiments, in addition to % monomer, HMW and LMW, peak areas can be recorded to calculate recovery from the SEC column. In some embodiments, column temperature for both UPLC and HPLC can be set to 30° C.

In some embodiments, a Labchip CE instrument (GX II) is used to evaluate one or more proteins. In some embodiments, samples are diluted to 1 mg/mL at each time point and mixed with Caliper running buffer with either dithiothreitol (reducing samples) or iodoacetamide (non-reducing samples). Samples can be run on a Protein Express Chip following the manufacturer's recommendations. Electropherograms can be analyzed by peak integration and HMW and LMW determination.

In some embodiments, Imaging capillary IsoElectric Focusing (iCIEF) can be used to evaluate one or more proteins. In some embodiments, iCIEF can be performed using an iCE280 analyzer (or similar) with a fluorocarbon coated capillary cartridge from Protein Simple. Methylcellulose, pI markers, and testing kits also can be purchased from Protein Simple. In some embodiments, the ampholyte solution contains 35% water, 2 M urea, 35% of 1.0% methylcellulose, 4% of pharmalyte, and 0.35% appropriate high and low pI markers. In some embodiments, the anolyte is 80 mM phosphoric acid, and the catholyte is 100 mM sodium hydroxide, both in 0.1% methylcellulose. Protein (e.g., 6 μg) can be mixed with 200 μL of ampholyte solution and then focused by introducing a potential of 1500 Volts (V) for 1 minute, followed by a potential of 3000 V for 8 minutes. An image of the focused charge variants can be obtained by passing 280 nm UV light through the capillary and into the lens of a charge coupled device digital camera. The chromatogram can be analyzed using Empower software to determine the distribution of the various charge variants.

In some embodiments, an interaction coefficient (kD) can be determined for one or more proteins. In some embodiments, samples are made up at pH 5.5 in 20 mM His, 150 mM Arginine at 20, 10, 5, 2.5 and 1.25 mg/mL. In some embodiments, the diffusion coefficient can be measured using a Wyatt DynaPro instrument. The kD can be determined by plotting diffusion coefficient against concentration.

In some embodiments, protein precipitation with Polyethylene Glycol (PEG) can be performed. In some embodiments, using 10× concentrated citrate buffer and a 40% PEG-8000 solution, material can be diluted to 1 mg/mL into 96-well plates in the presence of PEG-8000 from 0 to 36% in 48 steps. In some embodiments, plates are incubated at room temperature for 24 hours before being filtered using Corning filter plates. Of the resulting filtrate, 75 μL can be placed in a UV-transparent plate and the concentration can be measured. In some embodiments, data analysis can involve fitting a sigmoidal curve to a plot of the log of the concentration as a function of PEG concentration and reporting the PEG concentration at the mid-point of the sigmoidal. Alternatively, fitting the linear region and extrapolating back to 0% PEG can be used to evaluate the apparent solubility.

In some embodiments, Effective charge (Zeff) can be evaluated for one or more proteins. In some embodiments, samples are made up in their respective formulation buffers at 1.25 mg/mL. The electrophoretic mobility can be measured using a Wyatt Möbiζ instrument (e.g., 500-800 μL injection volume, Phase Analysis Light Scattering (PALS) collection period 10 s, voltage amplitude 3.5V, electric field frequency 20 Hz, “Henry” model). Zeta potential and effective charge can be calculated (e.g., automatically) from electrophoretic mobility.

In some embodiments, Hydrophobic Interaction Chromatography (HIC) is used to evaluate one or more proteins. In some embodiments, hydrophobicity is measured using a Waters ProteinPac HIC column. In some embodiments, Mobile phase A can be 20 mM sodium phosphate pH 6.5, 1.5 M ammonium sulfate. In some embodiments, Mobile phase B can be the same as A without ammonium sulfate. The sample can be prepared by diluting protein least 10 fold in 100% mobile phase A. The injection volume can be large enough to load at >2 μg onto the column. The column can be equilibrated with 100% A flowing at 1 mL/min with the column temperature set at 30° C. In some embodiments, after injection, the column can be washed for 2 minutes followed by a linear gradient from 100% to 0% mobile phase A over the course of 35 minutes. The column can be equilibrated with several column volumes of mobile phase A between injections.

In some embodiments, Liquid Chromatography-Mass Spectrometry (LCMS) can be used to evaluate one or more proteins.

In some embodiments, one or more properties are evaluated as a function of time. For example, the effect of a pH, buffer, and/or excipient on a biological product can be evaluated by measuring or otherwise determining one or more property levels as a function of time, for example by determining levels at one or more different time points (e.g., each separated by 1-24 hours, 1-7 days, or 1-4 weeks, or longer time points). In some embodiments, other factors such as temperature are set to stress the formulation and accelerate any change in one or more properties. For example, in some embodiments a formulation may be incubated at room temperature. However, in some embodiments, a formulation may be incubated below room temperature (e.g., from around 0 to 20° C., around 5° C., around 10° C., or around 15° C.) in order to provide an additional external stress on the formulation that may be useful to reveal one or more changes in the protein properties that could be informative for subsequent monitoring or formulation considerations. Similarly, in other embodiments a formulation may be incubated above room temperature (e.g., from around 25 to 60° C., around 35 to 55° C., around 40° C., around 45° C., around 50° C., around 55° C., or around 60° C.) in order to provide an additional external stress on the formulation that may be useful to reveal one or more changes in the protein properties that could be informative for subsequent monitoring or formulation considerations.

Accordingly, in one aspect, provided herein is a method for developing a biological therapeutic product comprising

a) evaluating a pH effect on one or more properties of at least one biological therapeutic agent and identifying an acceptable pH range within which the at least one biological therapeutic agent is stable;

b) evaluating an effect of two or more buffers on one or more properties of the at least one biological therapeutic agent within the acceptable pH range and selecting an acceptable buffer within which the at least one biological therapeutic agent is stable; and

c) evaluating an effect of two or more excipients on one or more properties of the at least one biological therapeutic agent within the acceptable pH range in the acceptable buffer and selecting one or more excipients upon which the at least one biological therapeutic agent is stable.

In certain embodiments, evaluation of the pH effect in step (a) comprises

(i) preparing a first batch of multiple solutions each comprising the at least one biological therapeutic agent at a pH from about 1.0 to 13.0 and initially evaluating the one or more properties;

(ii) incubating the first batch solutions for a first period of time and firstly evaluating the one or more properties; and

(iii) optionally further incubating the first batch solutions for a second period of time and evaluating the one or more properties.

In certain embodiments, evaluation of the buffer effect in step (b) comprises

(iv) preparing a second batch of multiple solutions with two or more buffers, wherein each solution comprises the biological therapeutic agent and a buffer at a pH within the acceptable pH range;

(v) initially evaluating the one or more properties of the biological therapeutic agent in each solution;

(vi) incubating the solutions of the second batch for a third period of time and evaluating the one or more properties of the biological therapeutic agent in each solution; and

(vii) optionally further incubating the solutions of the second batch for a fourth period of time and evaluating the one or more properties of the biological therapeutic agent in each solution.

In certain embodiments, evaluation of the excipient effect in step (c) comprises (viii) preparing a third batch of multiple solutions each comprising the biological therapeutic agent, the optimal buffer, and one or more excipients at the acceptable pH;

(ix) initially evaluating the one or more properties of the biological therapeutic agent in each solution;

(x) incubating the solutions of the third batch for a fifth period of time and evaluating the one or more properties of the biological therapeutic agent in each solution;

(xi) optionally further incubating the solutions for a sixth period of time and evaluating the one or more properties of the biological therapeutic agent in each solution.

In certain embodiments, the solutions of the third batch are subject to agitation stress for a fifth period of time and the one or more properties are evaluated. In certain embodiments, the solutions of the third batch after agitation stress for a fifth period of time are subject to agitation stress for a sixth period of time, and the one or more properties are evaluated.

In certain embodiments, the solutions of the third batch are subject to freeze-thaw stress for a fifth period of time and the one or more properties are evaluated. In certain embodiments, the solutions of the third batch after freeze-thaw for a fifth period of time are subject to freeze-thaw for a sixth period of time, and the one or more properties are evaluated.

The present invention adopts a systematic approach to evaluate formulation variables such as pH effect, buffer effect, and excipient effect on the one or more properties of the formulations.

In certain embodiments, the one or more properties evaluated are the physical stability. In certain embodiments, the properties evaluated are one or more selected from the group consisting of conformational stability, colloidal stability, interfacial stability, aggregation, and charge (pI). In certain embodiments, the properties evaluated are degradation and aggregation properties. In certain embodiments, the properties evaluated are conformation stability, protein integrity, aggregation, and charge (pI). In certain embodiments, the physical property evaluated is the conformation stability. In certain embodiments, the physical property evaluated is the protein integrity. In certain embodiments, the physical property evaluated is the aggregation. In certain embodiments, the physical property evaluated is the heterogeneities from charge isoforms. In certain embodiments, the one or more properties are evaluated with differential scanning calorimetry (DSC), size exclusion chromatography (SEC), gel electrophoresis (GXII), or charge variant analysis (icIEF) (J. Pharm. Sci., 2010, 99(5), 2279-2294).

In some embodiments, one or more properties of a protein of interest are evaluated in a pH spectrum from about 1.0 to about 12.0, from about 3.0 to about 9.0, or from about 4.0 to about 8.0. However, other pH ranges can be used. In some embodiments, a protein is evaluated under a plurality of different pH conditions within a pH range being studied (e.g., using pH solutions or buffers at 0.1, 0.2, 0.3, 0.4, 0.5, 0.6, 0.7, 0.8, 0.9, 1.0 or other pH unit intervals within a pH range of interest).

In some embodiments, the effects of different buffering agents can be evaluated. In some embodiments, a buffering agent is selected from the group consisting of, phosphate buffers, citrate buffers, succinate buffers, Tris buffers, glycine buffers, histidine buffers, or combinations of two or more thereof.

In some embodiments, one or more candidate molecules are studied to evaluate whether one or more of their biophysical and/or physico-chemical properties are likely to be challenges for pharmaceutical development (e.g., likely to result in unacceptable levels of protein modification, degradation, aggregation or other unwanted property in a pharmaceutical formulation). In some embodiments, a sequence analysis is performed to identify potential hotspots for modification or degradation. In some embodiments, a sequence analysis is performed on one or more Complementary Determining Regions (CDRs) of a therapeutic protein or candidate molecule (e.g., a therapeutic antibody or candidate antibody).

In some embodiments, the diffusion interaction parameter (kD) of a protein preparation can be measured by dynamic light scattering (DLS) as an indicator of colloidal and thermal stability. In some embodiments, DLS can be performed at pH 5.5 (20 mM His, pH 5.5).

In some embodiments, PEG precipitation can be performed (e.g., at pH 5.5 (20 mM His, pH 5.5)).

In some embodiments, one or more additional assays may include Hydrophobic interaction chromatography (HIC), viscosity determination (e.g., at 150 mg/mL), chemical denaturation, effective charge (e.g., Mobius) analysis. In some embodiments, a low pH hold (e.g., hold at pH 3 for 5 hours, neutralize and run on SEC) is performed.

In some embodiments, pH, buffer, and excipient screening are performed. For example, initially the effect of pH on stability and rate of chemical modification is evaluated. In some embodiments, the effects are evaluated at several different pHs (e.g., 4, 5, 6, 7, and 8). In some embodiments, the effects of several different buffers are evaluated (e.g., citrate, phosphate, and/or histidine). In some embodiments, accelerated stability assays are performed (e.g., by incubating the one or more proteins at 40° C. for 2 weeks). In some embodiments, the effects of different buffers are then evaluated (for example, using a pH or selected pH range at which the protein is relatively stable). In some embodiments, the effects of different excipients are then evaluated (for example, using a pH and/or a buffer in which the protein is relatively stable). In some embodiments, the effects of different excipients are evaluated at relatively high excipient concentrations, for example, between 0.01% (w/v) and 10% (w/v) depending on the excipient (e.g., using a surfactant at between 0.01% to 0.1% or higher, using sucrose or similar excipient at greater than 1%, 2%, 3%, 4%, 5%, 6%, 7%, 8%, 9%, and up to 10% (=300 mM) or higher, one or more buffers at 10-20 mM or higher (for example, up to 50 mM, 50-100 mM, or higher), one or more salts (e.g., NaCl) at 50-150 mM, or up to 200 mM, or higher, one or more amino acids (e.g., Arg) at 50-150 mM, or up to 200 mM, or higher, and/or other excipients or any combination of two or more thereof).

In some embodiments, one or more assays can be performed to evaluate the stability of the protein(s). Non-limiting examples of assays include SEC (e.g., to evaluate the formation of high molecular weight (HMW) or low molecular weight (LMW) products), iCIEF (e.g., to evaluate changes in charged isoforms, reducing and/or native Capillary Electrophoresis (CE) or GXII (e.g., to determine clipping and/or loss of one or more protein subunits, for example loss of light chain from a therapeutic antibody or antibody candidate), LC-MS (e.g., to evaluate deamidation, isomerization, and/or oxidation (e.g., gated on iCIEF and sequence analysis)), and/or Differential Scanning calorimetry (e.g., to determine thermal stability and/or the onset of unfolding.

Protein evaluation studies (e.g., under different pH conditions, in different buffers, and/or in the presence of different excipients) can be performed at any suitable protein concentration. In some embodiments, protein concentrations at 10 mg/mL are used, but lower concentrations (e.g., around 0.5 mg/mL, around 1 mg/mL, or other concentrations) also may be evaluated. In some embodiments that are evaluating protein stability at high concentration, protein concentrations over 50 mg/mL (e.g., up to or around 100 mg/mL, up to or around 150 mg/mL, up to or around 200 mg/mL, up to or around 250 mg/mL, or up to or around 300 mg/mL, or intermediate or higher concentrations) may be evaluated.

In some embodiments, assays are performed at a series of time points (e.g., daily, or weekly, for example at zero, one, and two weeks) for proteins that are exposed to conditions that can accelerate changes (e.g., incubation above room temperature, for example at about 30° C., 35° C., 40° C., 45° C., 50° C., 55° C., 60° C., or other suitable temperature depending on the protein).

In some embodiments, a candidate protein can be selected based on a comparison of its stability to other candidate proteins. For example, the most stable protein amongst a plurality of tested proteins may be selected. In some embodiments, protein stability is not the only factor being considered and the relative stability of a candidate protein can be one of several factors (for example in addition to other factors such as pharmacokinetic or pharmacodynamics properties, potential toxicity, side effects, cost, etc.) that can be considered when selecting a protein candidate for pharmaceutical formulation. Accordingly, in some embodiments a candidate protein that is selected may not be the most stable amongst a plurality of tested candidates, but it may be sufficiently stable (e.g., relative to other candidates) to be selected in view of its other properties. In some embodiments, a candidate protein is compared to a reference protein (e.g., a reference therapeutic protein that is sufficiently stable in pharmaceutical formulations) to determine whether the candidate is sufficiently stable for subsequent pharmaceutical formulation. In some embodiments, if a protein that is selected for further formulation (e.g., based on factors in addition to the stability and solubility of the protein under the evaluated conditions) is not the most stable or soluble of the proteins that were tested, then information from the stability and/or solubility studies can be used to help select or develop a suitable formulation for that protein even if the conditions are different from the ones that were used to evaluate the protein. For example, if the protein is identified, as a result of the evaluation, to be susceptible to certain chemical modifications, degradation, aggregation, or other change that may not be pharmaceutically desirable, then these tendencies or susceptibilities can be taken into account when selecting or developing a suitable formulation for the protein.

Similarly, in some embodiments different potential formulation conditions can be compared to determine which one is the best (or sufficient) for formulating a therapeutic protein.

In some embodiments, guidelines for selecting candidate therapeutics and/or formulation conditions can be used. For example, in some non-limiting embodiments, a candidate protein and/or formulation condition is acceptable if the Tm1 onset at pH 5-7 is >around 50-60° C. (e.g., >55° C., >60° C., or higher). In some embodiments, lower hydrophobicities (e.g., measured using HIC) are better. In some embodiments, a KD at pH 5.5 (His+/−Argcl) of >−5 to −10 (e.g., >−8) is acceptable. In some embodiments, a pI>6-8 (e.g., pI>7) is acceptable. In some embodiments, higher PEG solubility mid-points at pH 5.5 are better. In some embodiments, viscosity at 150 mg/mL (with or without Arginine)≤about 10-20 cP (e.g., ≤about 12-18 cP, for example ≤12 cP, ≤13 cP, ≤14 cP, ≤15 cP, ≤16 cP, ≤17 cP, or ≤18 cP) is acceptable. In some embodiments, accelerated stability (e.g., at ≥100 mg/ml) is evaluated, and one or more of the following non-limiting guidelines can be used as acceptable: a ΔHMW after 4 weeks at 40° C. at any pH 4-8≤about 5-10%, ≤about 1-5% (for example, ≤1%, ≤2%, ≤3%, ≤4%, or ≤5%), a ΔDegradation after 4 weeks at 40° C.≤about 5-10%, ≤about 1-5% (for example, ≤1%, ≤2%, ≤3%, ≤4%, or ≤5%), a ΔDeamidation (e.g., in CDRs)≤5-15% (for example, ≤5%, ≤6%, ≤7%, ≤8%, ≤9%, ≤10%, ≤11%, ≤12%, ≤13%, ≤14%, or ≤15%), a ΔIsomerization (e.g., in CDRs)≤5-15% (for example, ≤5%, ≤6%, ≤7%, ≤8%, ≤9%, ≤10%, ≤11%, ≤12%, ≤13%, ≤14%, or ≤15%), and/or a relatively low ΔOxidation. These are examples of non-limiting thresholds and other thresholds can be used for different proteins and/or formulations (for example, depending on the protein, the concentration, the intended use, etc.).

In some embodiments, pH conditions, buffers, and excipients that are identified or selected using methods described in this application may be used to formulate a protein of interest. In some embodiments, the pH, buffer and excipient concentrations that are tested can be used for the final pharmaceutical formulation. In some embodiments, the pH, buffer and excipient concentrations that are tested can be used as a starting point for further refinement in subsequent formulation studies (e.g., to optimize one or more components for a final pharmaceutical formulation). In some embodiments, additional components can be included in a final pharmaceutical formulation.

In some embodiments, the behavior of a therapeutic protein product of interest under different pH, buffer, and excipient conditions can be used, for example, to identify potential stability issues that a selected protein may present upon formulation. This can be helpful to determine how best to formulate the protein of interest. In some embodiments, the protein of interest may not be formulated under the conditions that were tested, but the results from the tested conditions can be used to help determine suitable formulation conditions to evaluate and develop in order to address one or more tendencies (e.g., degradation, chemical modification, aggregation, etc., or any combination thereof) of the protein of interest.

As used herein, an excipient is a pharmaceutically acceptable excipient used in the manufacture of pharmaceutical compositions. Pharmaceutically acceptable excipients include inert diluents, dispersing and/or granulating agents, surface active agents and/or emulsifiers, disintegrating agents, binding agents, preservatives, buffering agents, lubricating agents, and/or oils. In certain embodiments, one excipient is present. In certain embodiments, one or more excipients are present. In certain embodiments, one or more excipients are Generally Recognized as Safe (GRAS) excipients. In certain embodiments, one or more excipients are selected from the group consisting of inorganic salts, sugars, polyols, amino acids, and polymers.

Exemplary inorganic salts include, but are not limited to, IA and IIA metal halides (e.g., NaCl, MgCl₂, or KCl). Exemplary sugars include, but are not limited to trehalose, mannose, sucrose, and dextrose. Exemplary polyols include, but are not limited to sorbitol, mannitol, and glycerol. Exemplary amino acids include, but are not limited to, arginine, histidine, aspartic acid, alanine, and glutamic acid. Exemplary polymers include, but are not limited to polysorbate, albumin, and gelatin.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

In certain embodiments, the concentrations of the biological therapeutic agent in the first batch solutions are about 0.01 to about 10 mg/mL. In certain embodiments, the concentrations of the biological therapeutic agent in the first batch solutions are about 0.1 to about 10 mg/mL. In certain embodiments, the concentrations of the biological therapeutic agent in the first batch solutions are about 0.5 to about 5 mg/mL. In certain embodiments, the concentrations of the biological therapeutic agent in the first batch solutions are about 0.5 to about 3 mg/mL. In certain embodiments, the concentrations of the biological therapeutic agent in the first batch solutions are about 1 mg/mL.

In certain embodiments, the concentrations of the biological therapeutic agent in the second batch solutions are about 0.01 to about 10 mg/mL. In certain embodiments, the concentrations of the biological therapeutic agent in the second batch solutions are about 0.1 to about 10 mg/mL. In certain embodiments, the concentrations of the biological therapeutic agent in the second batch solutions are about 0.5 to about 5 mg/mL. In certain embodiments, the concentrations of the biological therapeutic agent in the second batch solutions are about 0.5 to about 3 mg/mL. In certain embodiments, the concentration of the biological therapeutic agent in the second batch solutions are about 1 mg/mL.

In certain embodiments, the concentrations of the biological therapeutic agent in the third batch solutions are about 0.01 to about 100 mg/mL. In certain embodiments, the concentrations of the biological therapeutic agent in the third batch solutions are about 0.1 to about 80 mg/mL. In certain embodiments, the concentrations of the biological therapeutic agent in the third batch solutions are about 1.0 to about 70 mg/mL. In certain embodiments, the concentrations of the biological therapeutic agent in the third batch solutions are about 10 to about 60 mg/mL. In certain embodiments, the concentrations of the biological therapeutic agent in the third batch solutions are about 0.1 to about 10 mg/mL. In certain embodiments, the concentrations of the biological therapeutic agent in the third batch solutions are about 0.5 to about 5 mg/mL. In certain embodiments, the concentrations of the biological therapeutic agent in the third batch solutions are about 0.5 to about 3 mg/mL. In certain embodiments, the concentrations of biological therapeutic agent in the third batch solutions are about 50 mg/mL. In certain embodiments, the concentrations of biological therapeutic agent in the third batch solutions are about 1 mg/mL when subjecting to agitation stress.

In certain embodiments, the initial pH evaluation is carried out in a pH spectrum from about 1.0 to about 12.0. In certain embodiments, the initial pH evaluation is carried out in a pH spectrum from about 2.0 to about 11.0. In certain embodiments, the initial pH evaluation is carried out in a spectrum from about 3.0 to about 9.0. In certain embodiments, the initial pH evaluation is carried out in a spectrum from about 4.0 to about 9.0. In certain embodiments, the initial pH evaluation is carried out in a spectrum from about 5.0 to about 8.0.

In certain embodiments, the first period of time is about a week. In certain embodiments, the second period of time is about a week. In certain embodiments, the third period of time is about a week. In certain embodiments, the fourth period of time is about a week. In certain embodiments, the fifth period of time is about a week. In certain embodiments, the sixth period of time is about a week.

As used herein, a biological therapeutic agent refers to any medicinal product manufactured in or extracted from biological sources. Examples of biological therapeutic agents include, but are not limited to, vaccines, blood or blood components, allergenics, somatic cells, gene therapies, tissues, recombinant therapeutic protein and living cells. In certain embodiments, the biological therapeutic agent is wild type. In certain embodiments, the biological therapeutic agent is engineered. In certain embodiments, the biological therapeutic agent is a protein. In certain embodiments, the biological therapeutic agent is an antibody. In certain embodiments, the biological therapeutic agent is a B cell antibody. In certain embodiments, the biological therapeutic agent is a T cell antibody. In certain embodiments, the biological therapeutic agent is a cytokine.

In certain embodiments, the provided methods are to formulate at least one biological therapeutic agent. In certain embodiments, the provided methods are to screen at least one biological therapeutic agent for a biological therapeutic product candidate. In certain embodiments, the selected biological therapeutic product candidate has one or more acceptable properties in the second and/or third batch solutions. In some embodiments, the acceptable properties of the biological therapeutic candidates are the acceptable physical stability of the biological therapeutic agent. In some embodiments, the acceptable stability of the selected biological therapeutic agent generally has minimum aggregation in the first, second, and third batch solutions. In some embodiments, the acceptable stability of the selected biological therapeutic agent generally has minimum aggregation in the second and/or third batch solutions. In some embodiments, the acceptable stability of the selected biological therapeutic agent generally has minimum degradation in the first, second, and third batch solutions. In some embodiments, the acceptable stability of the selected biological therapeutic agent generally has minimum degradation in the second and/or third batch solutions. It is to be understood that the selected biological therapeutic candidate with the acceptable stability generally has a better stability and degradation profile compared to other biological therapeutic agents, although it can be outperformed by another biological therapeutic agent at one or two measurements.

It is to be understood that the provided methods (e.g., for formulation and/or screening purpose) can be carried out in a high throughput fashion. In certain embodiments, provided methods are carried out on an automated system. The automated system may be constructed to perform sample preparation, testing, and data management. In certain embodiments, the instrumentation utilized is not altered in capability or use and is readily available for purchase. However, each procedure can optimized for material conservation while maintaining data quality. These updated procedures can be incorporated into workflows crafted specifically for the purpose of utilizing very low volumes and biological therapeutic agent content. In certain embodiments, each candidate is evaluated in depth by Size Exclusion Chromatography, Imaged Capillary Isoelectric focusing charge analysis, GXII Lab Chip Chromatography, and Differential Scanning calorimetry.

In some aspects, the present disclosure provides methods for developing pharmaceutical formulations with a viscosity that is suitable for clinical use. As used herein, the term “viscosity” refers to a measure of fluids resistance to deformation, e.g., by a shear force or tensile force. Viscosity is often measured by a viscometer and expressed in centipoise (cP) units. In some embodiments, fluids having relative high viscosity display higher resistance to shear forces (e.g., are “thicker”) and thus under a particular force will flow more slowly than fluid having relatively low viscosity.

In some embodiments, order for a pharmaceutical formulation to be useful for injection (e.g., subcutaneous injection), its viscosity must be low enough for the solution to flow easily through a needle or other similar conduit or orifice without requiring an excessively high force. In some embodiments, the pharmaceutical formulation's viscosity is less than 100 centipoise (cP). In some embodiments, the pharmaceutical formulation's viscosity is in a range of 1 cP to 100 cP. In some embodiments, the pharmaceutical formulation's viscosity is in a range of 10 cP to 50 cP. In some embodiments, the pharmaceutical formulation's viscosity is in a range of 15 cP to 35 cP. In some embodiments, the pharmaceutical formulation's viscosity is 15 cP, 16 cP, 17 cP, 18 cP, 19 cP, 20 cP, 21 cP, 22 cP, 23 cP, 24 cP, 25 cP, 26 cP, 27 cP, 28 cP, 29 cP, 30 cP, 31 cP, 32 cP, 33 cP, 34 cP, 35 cP, 36 cP, 37 cP, 38 cP, 39 cP, or 40 cP. It should be appreciated that the viscosity of a liquid formulation can be measured using any suitable technique including using a rheometer, a viscometer (e.g., a capillary or microcapillary viscometer), or any other suitable device.

In some embodiments, a formulation has a viscosity suitable for injection through a needle or other suitable device (e.g., a catheter) in a range of 27 gauge to 31 gauge (e.g., 29 gauge to 31 gauge) in size. In some embodiments, the formulation has a viscosity suitable for injection through a needle or other suitable device having an inner diameter of 0.22 mm, 0.20 mm, 0.18 mm, 0.16 mm, 0.14 mm, 0.12 mm, or 0.10 mm. In some embodiments, the formulation has a viscosity suitable for injection through a needle or other suitable device having an inner diameter in a range of 0.05 mm to 0.50 mm, 0.10 mm to 0.30 mm or 0.10 mm to 0.20 mm. In some embodiments, the formulation has a viscosity suitable for injection through a needle or other suitable device in a range of 27 gauge to 31 gauge (e.g., 29 gauge to 31 gauge) in size while at room temperature (for example from 15-30° C., e.g., between 20° C. and 25° C.).

In some embodiments, formulation has a viscosity of less than 100 cP, e.g., less than 50 cP, at protein concentrations of up to 150 mg/mL (e.g., from 50-100 mg/mL or 100 to 150 mg/mL) or above 150 mg/mL (e.g., from 150 to 250 mg/mL, from 250 to 350 mg/mL, from 350 to 450 mg/mL, or above).

It should be appreciated that once appropriate liquid formulations are identified, in some embodiments dry (e.g., dry powder) components can be provided for reconstitution prior to use or administration. Also, in some embodiments suitable liquid formulations can be partially dried or lyophilized (e.g., for storage or transport) and reconstituted into a liquid formulation having the appropriate volume (and related concentrations of components) prior to further use (e.g., prior to administration to a patient).

### EXAMPLES

Aspects of the disclosure are useful to evaluate one or more protein properties for formulation using a relatively small amount of protein. By systematically evaluating different formulation characteristics (e.g., pH, buffer, and excipients) independently of each other, less protein can be used that would be required to evaluate all combinations of pH, buffer, and excipients. In the following examples, about 50-100 mg of several protein candidates (e.g., monoclonal antibodies) were specifically formulated and evaluated for degradation and aggregation properties under different conditions as described herein. The data were collected by Size Exclusion Chromatography, Imaged Capillary Isoelectric focusing charge analysis, GXII Lab Chip Chromatography, and Differential Scanning calorimetry.

### Example 1. Evaluation of pH Effect

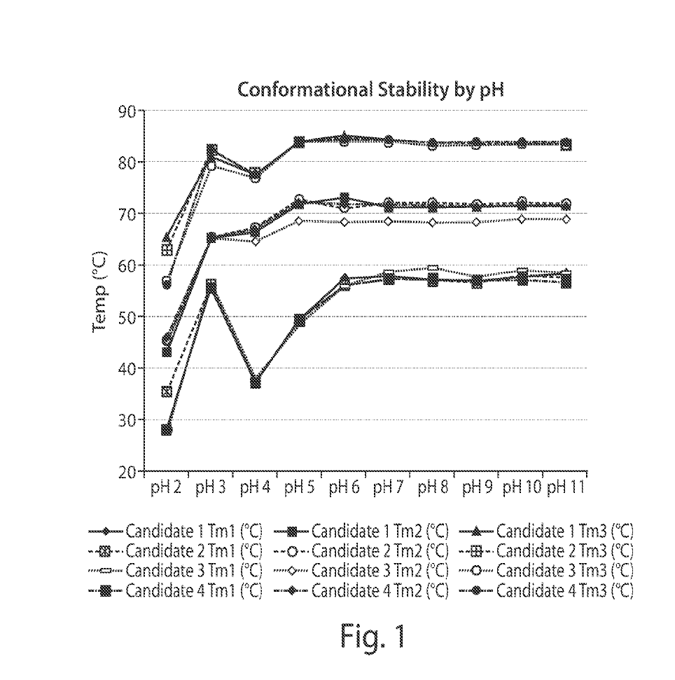

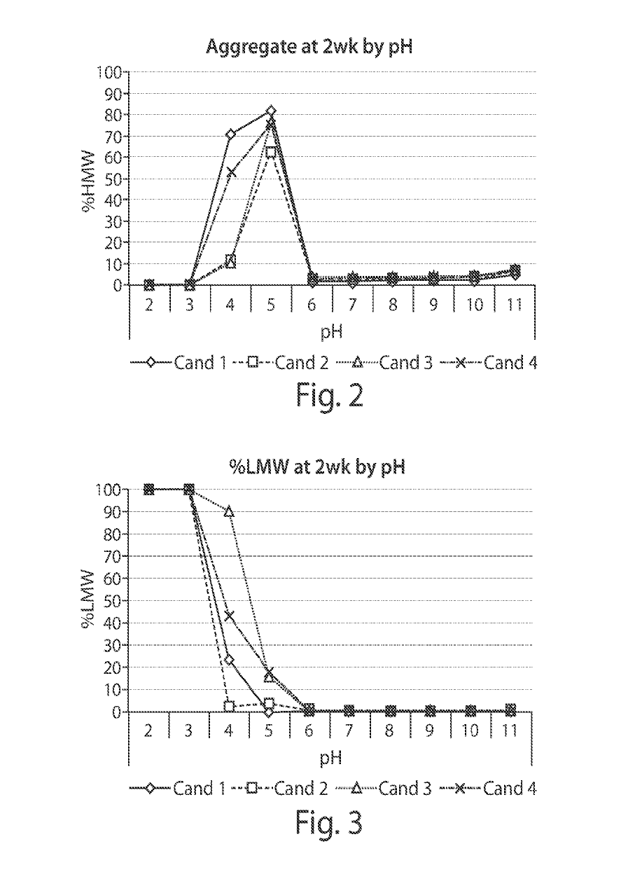

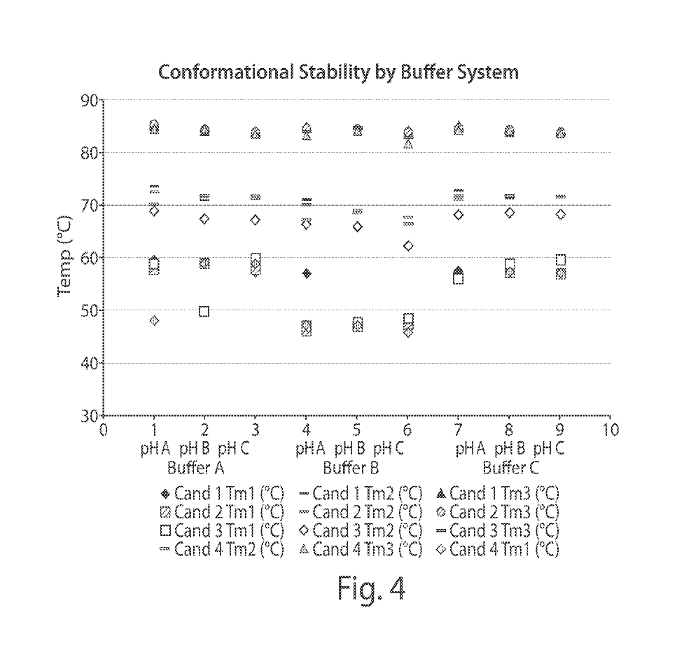

The behavior of four candidate proteins is evaluated for a wide pH range from pH 2 to pH 11 (FIGS. 1-3). The pH effect was evaluated by incubating the candidate proteins at different pHs (e.g., at different pHs in citrate buffers titrated at different pHs from pH 2 to pH 11). There was lower instability at the lower pH values studied. Candidate 3 appears to have a greater risk of conformation issue due to instability in the Fab region. It indicates that conformational stability may be a limiting factor in this program (e.g., for these candidate proteins). These results are helpful in indicating a pH window amenable for stability (e.g., for further analysis with different buffers and/or excipients). These results also are useful for identifying properties of the proteins to monitor when evaluating the effect of other factors such as different buffers and/or excipients in subsequent or separate experiments.

### Example 2. Evaluation of Buffer Effect

Based on the evaluation of pH effect, three pH values, 6, 7, and 8, were selected to formulate the four candidate proteins in three buffers (phosphate, His, and citrate).

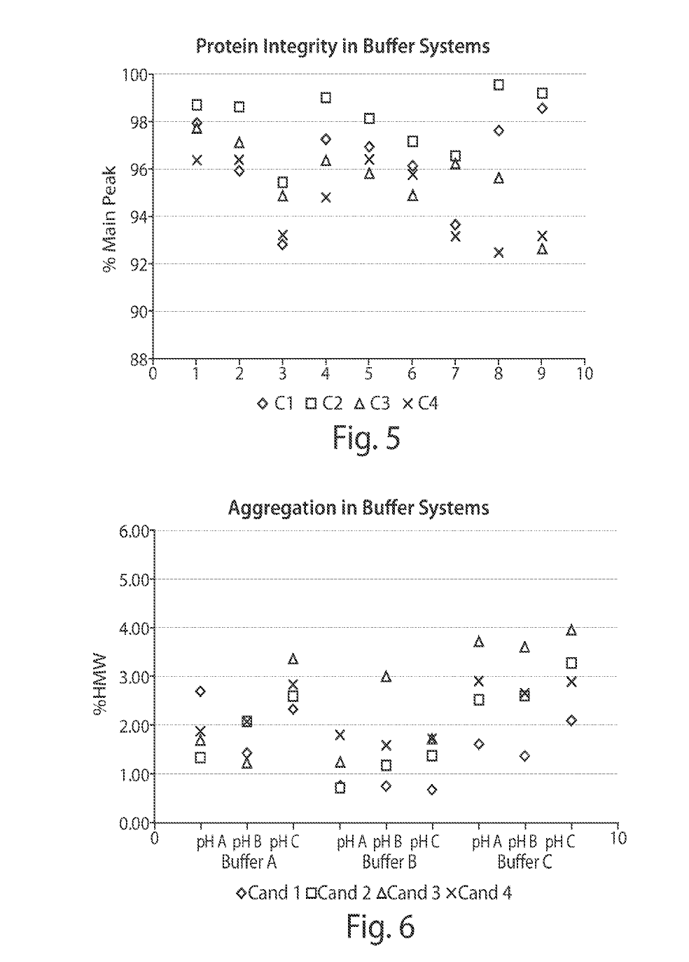

DSC analysis indicates that Buffer B appears to reduce conformational stability across candidates. Within the three buffer, pH has no obvious on conformation retention. Candidate 3 continues to exhibit lower Fab conformational stability across buffers (FIG. 4).

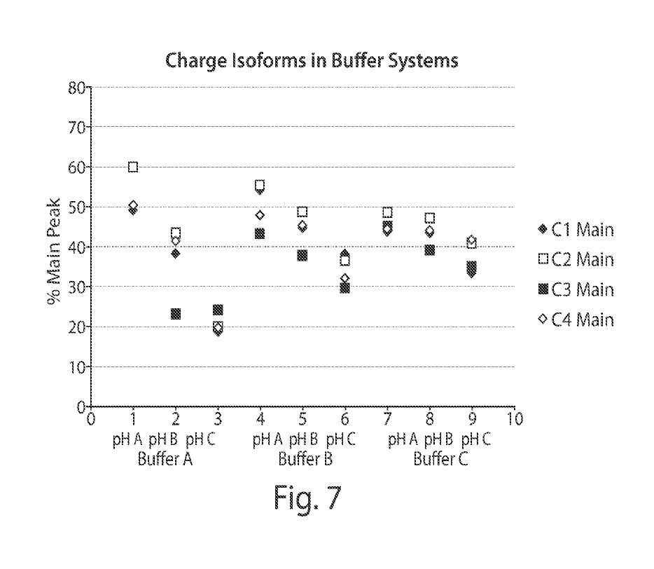

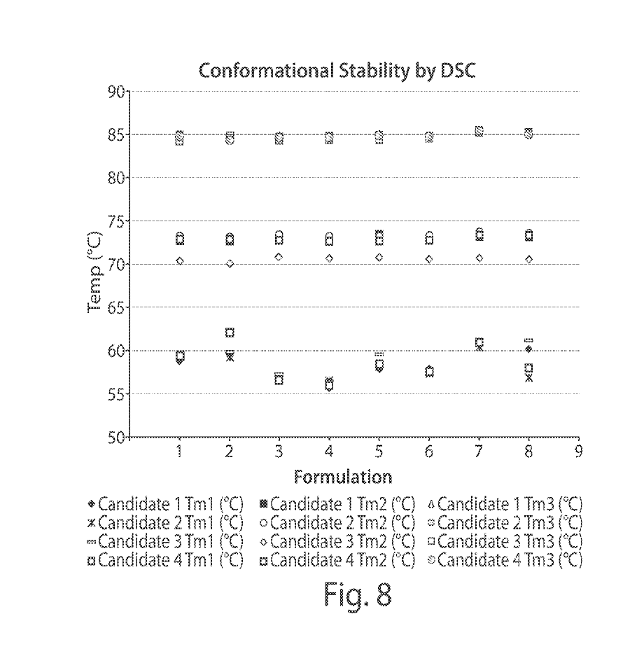

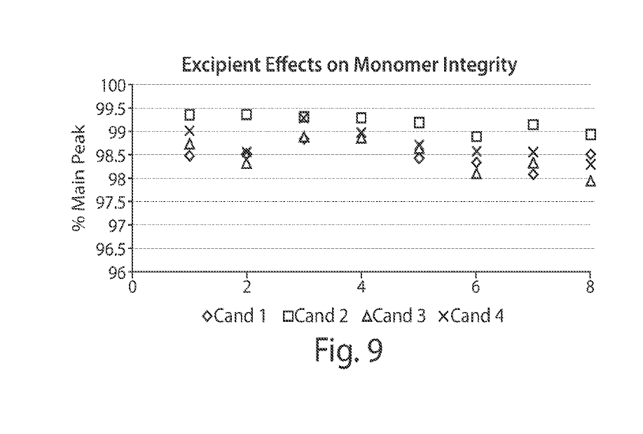

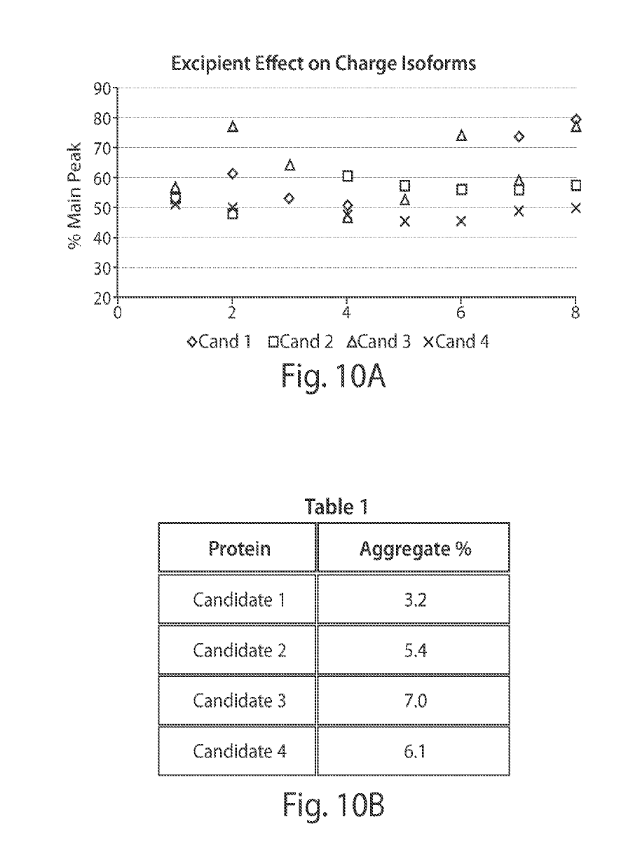

A pH-dependent relationship appears to exist in all buffers in the protein integrity, aggregation, and charge isoform analysis, though less pronounced in Buffer C (FIGS. 5-8). This relationship correlates increasing pH with loss of monomer integrity, increasing heterogeneity, and growth in acidic species. In several of the conditions, Candidate 2 retains higher amount of monomer, while Candidate 3 contains consistently lower aggregate levels. Buffer B appears to offer a greater protection against aggregation.

### Example 3. Evaluation of Excipient Effect

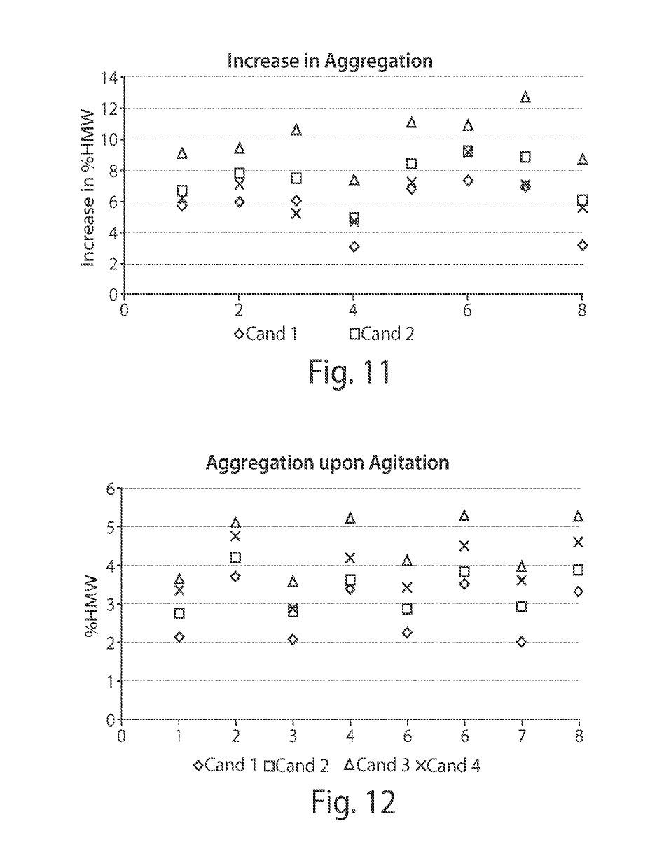

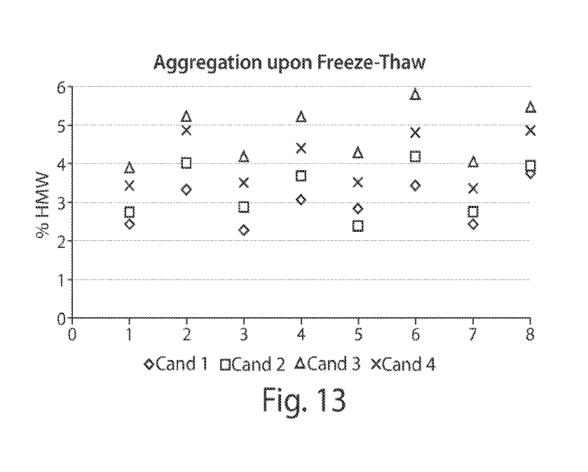

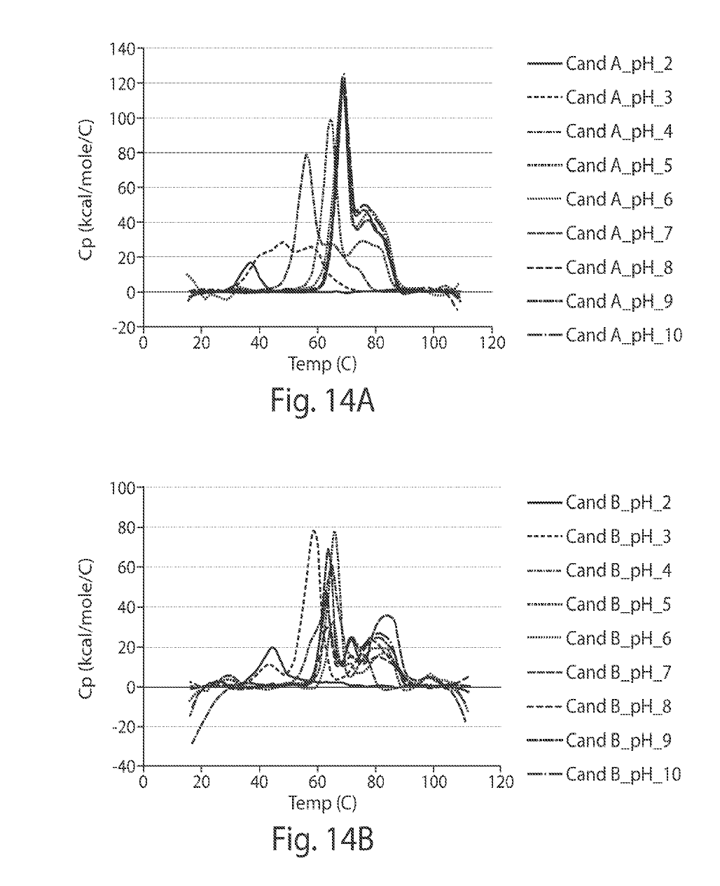

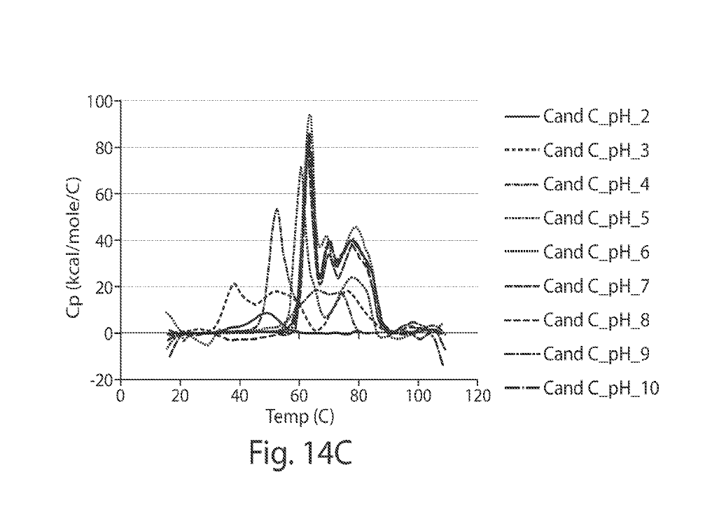

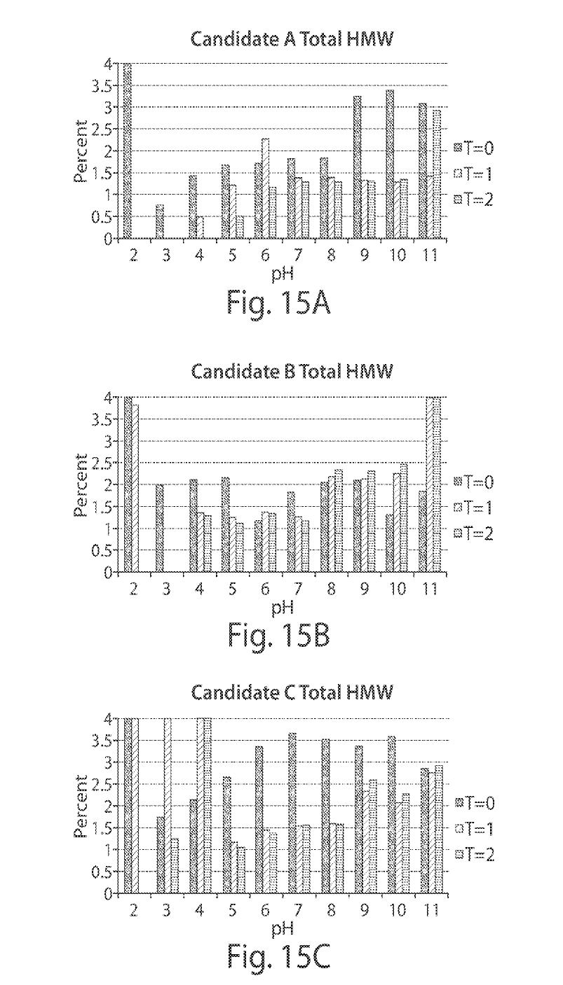

Based on the pH and buffer evaluations (Examples 1 and 2), pH 6.5 and the citrate buffer were selected to formulate the four candidate proteins with one or more excipients. Excipient effects on Monomer Integrity was measured by GXII Lab Chip analysis of protein solutions held in accelerated conditions for 2 weeks (FIG. 9). Excipient effect on charge isoforms was measured by iCIEF analysis of protein solutions held in accelerated conditions for 2 weeks (FIG. 10). At high concentrations, significant aggregation was observed for all four candidates in all basic formulations (FIG. 11). Aggregation of candidate proteins upon agitation and freeze-thaw does not shown significant effect on the studied excipients (FIGS. 12-13).

### Example 4. Protein Candidates Comparison

Four final candidates were compared. These were >98% identical, the only differences are a few mutations related to the humanization of these constructs. A summary of the comparison is shown in Table 2. Protein candidate 1 at pH A or B (pH 7 or 8) in Buffer B or C (His or citrate) with excipient sucrose and/or Arg appears to provide acceptable formulations for further development.

TABLE 2
Comparison of four protein candidates

| Aspect | Candidate 1 | Candidate 2 | Candidate 3 | Candidate 4 |
|---|---|---|---|---|
| Conformational | | | Tm2 lower | |
| Stability (DSC) | | | than others | |

Tm1 good above break point pH
Buffer B destabilizing, Buffer A = Buffer C
Formulation 3/4, 5/6 slightly destabilizing, 7/8 neutral
No consistent effect of adding surfactant observed

| | |
|---|---|
| Charge | More |
| varients | hetero- |
| (iCIEF) | geneity |

| | Optimal range identified in pH data | |
|---|---|---|
| | Decrease in main peak, increase in acidic peak with pH | |
| | Buffer C shows least variation with pH | |
| Degradation | More robust | |
| (GXII) | at low pH | |
| | Most degradation at low pH | |
| | No strong excipient effects | |
| Aggregation | Lowest % | Less |
| (SEC) | HMW | sensitive to |
| | | excipients? |

| | |
|---|---|
| | Buffer B slightly better than Buffer C, A |
| | Formulation 4/8 slightly stabilizing at high concentration |
| Agitation | None of the candidates appear overly sensitive |
| F/T | |

### Example 5. Preformulation

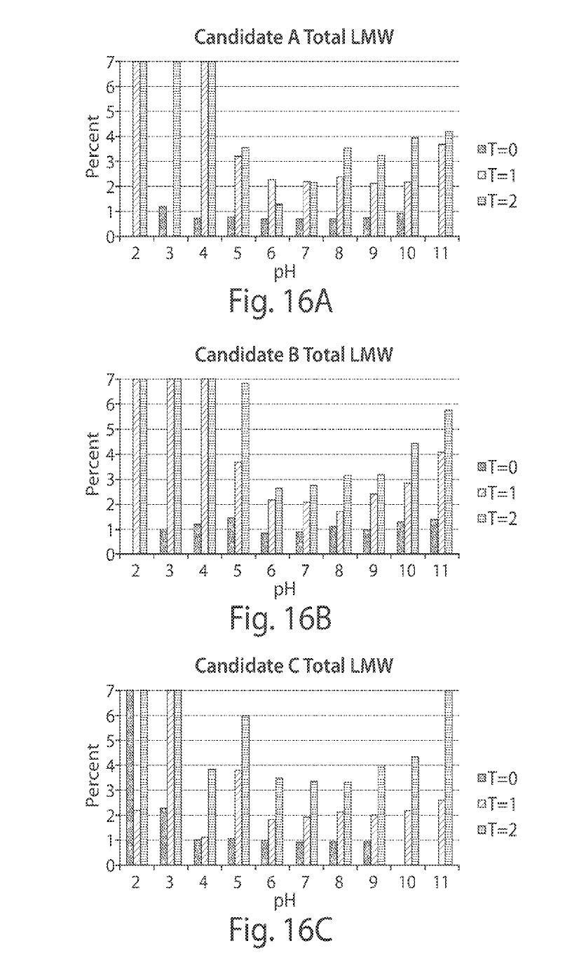

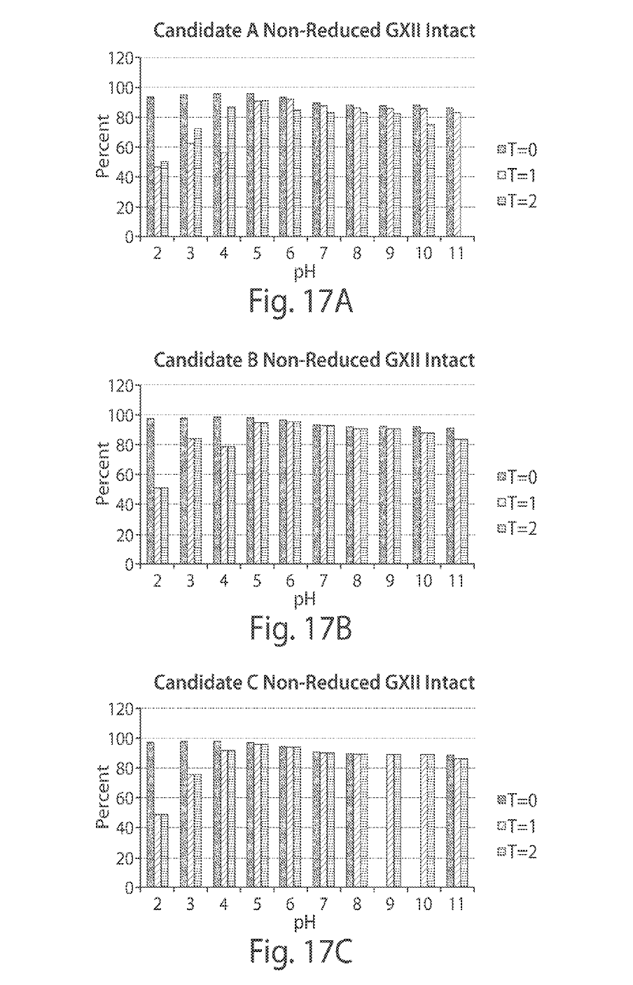

The behavior of three candidate proteins was evaluated for a wide pH range from pH 2 to pH 11 (FIGS. 14A-16C). The pH effect was evaluated by incubating the candidate proteins at different pH values (e.g., formulated in citrate buffers titrated at different pH values ranging from pH 2 to pH 11). DSC was used to examine the unfolding curves of the three candidate proteins. All candidates were generally stable above pH 5; at pH levels above 5, the onset temperature was greater than 60° C. (FIGS. 14A-14C).

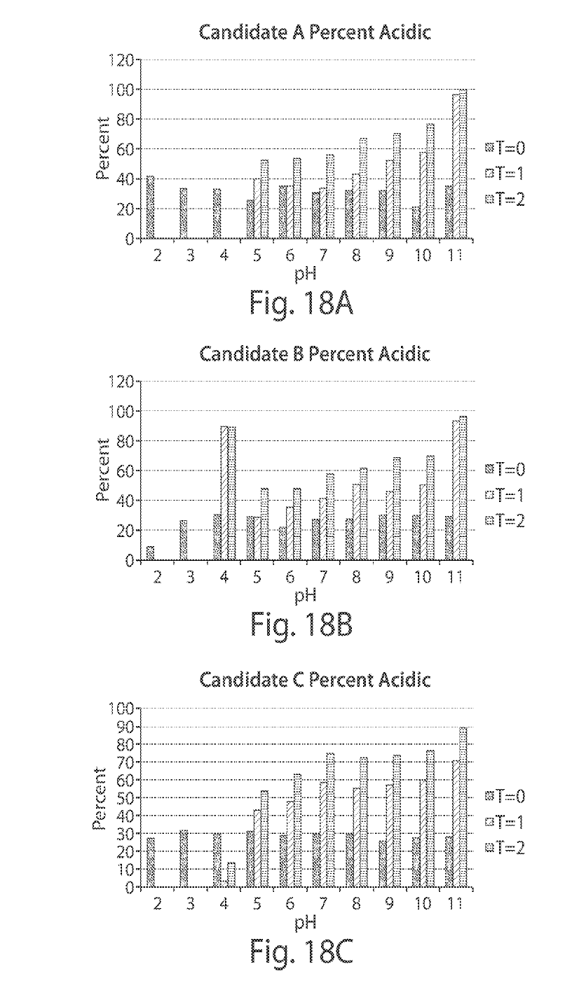

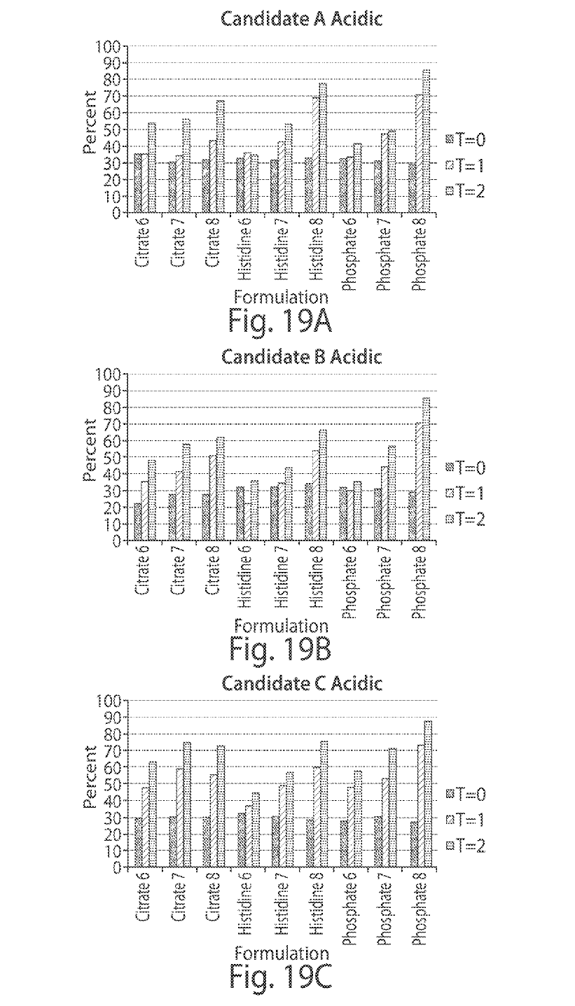

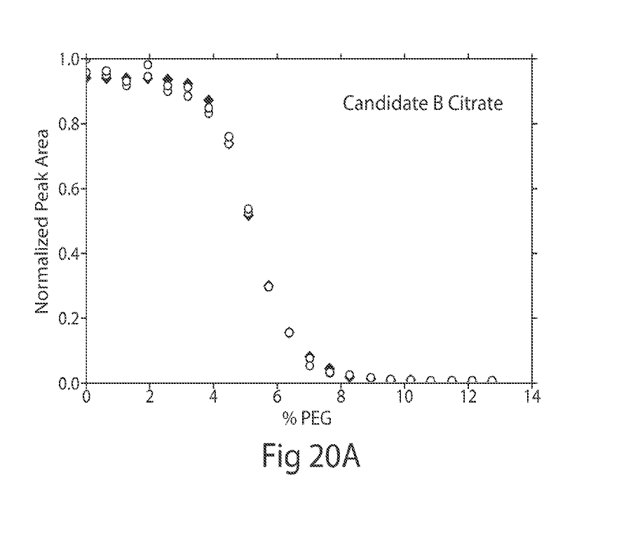

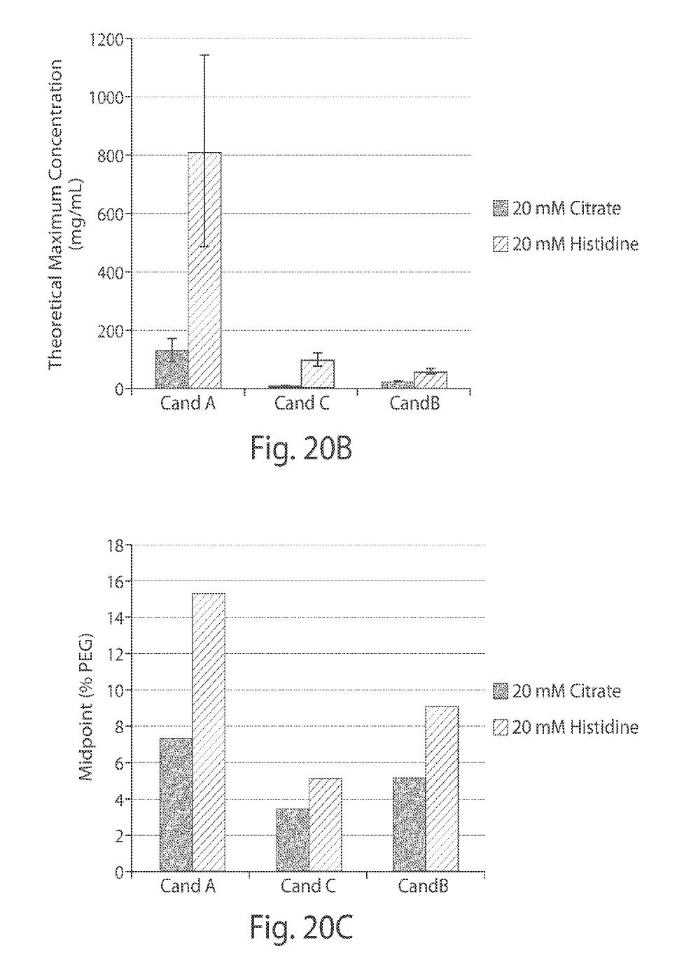

All three candidates were thermally stable between pH 5 and 8. Aggregation increased above pH 8 (FIGS. 15A-15C), while LMW formation increased below pH 5 (FIGS. 16A-16C). The formation of acidic or other chemically modified species may be the main form of degradation (FIGS. 17A-18C).

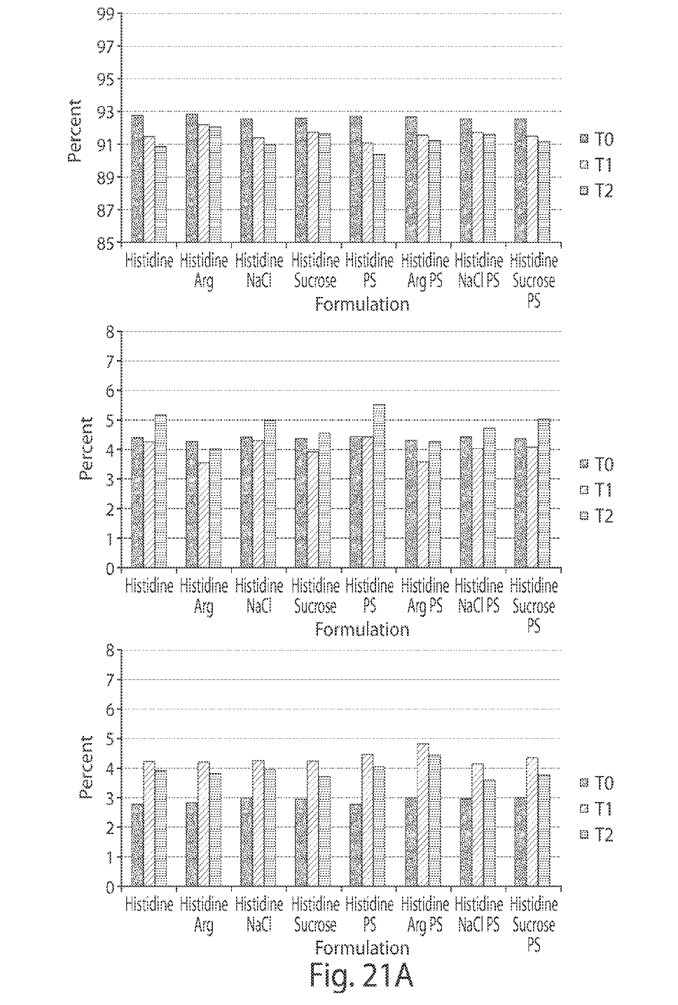

Buffer screens were performed using histidine, phosphate, and citrate between pH levels of 6 to 8 and 1 mg/mL of the candidate proteins. All candidate proteins behaved similarly under SEC. In an imaging Capillary IsoElectric Focusing (iCIEF) analysis on all three candidates, phosphate appeared to be the worst buffer, and there was little difference between citrate and histidine (FIGS. 19A-19C). Also, pH levels between 6 and 7 seemed to be the best.

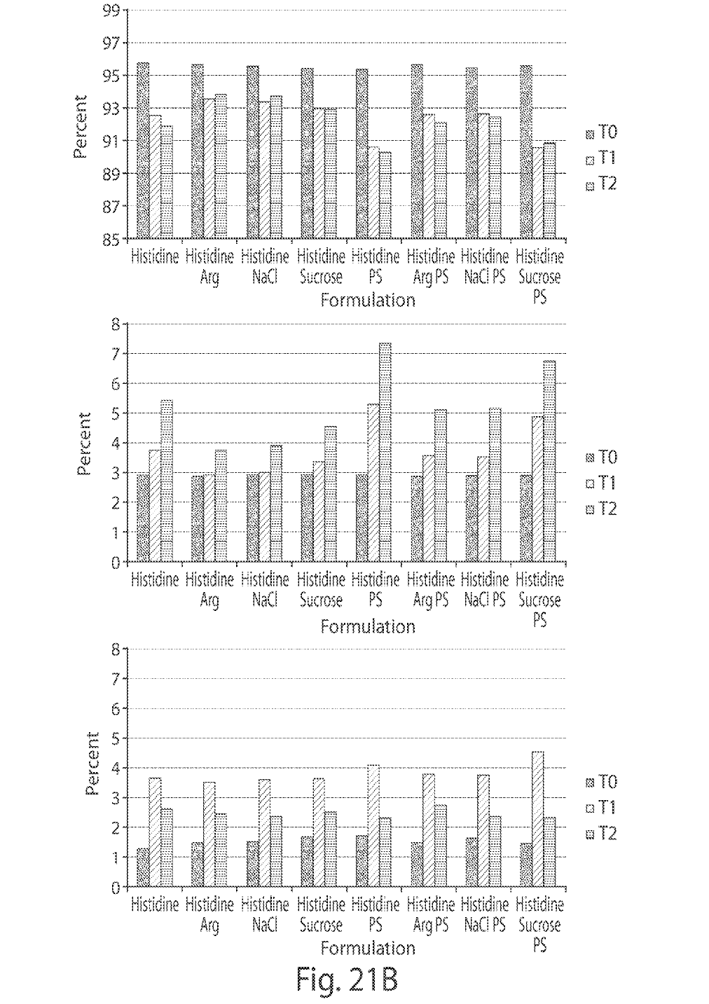

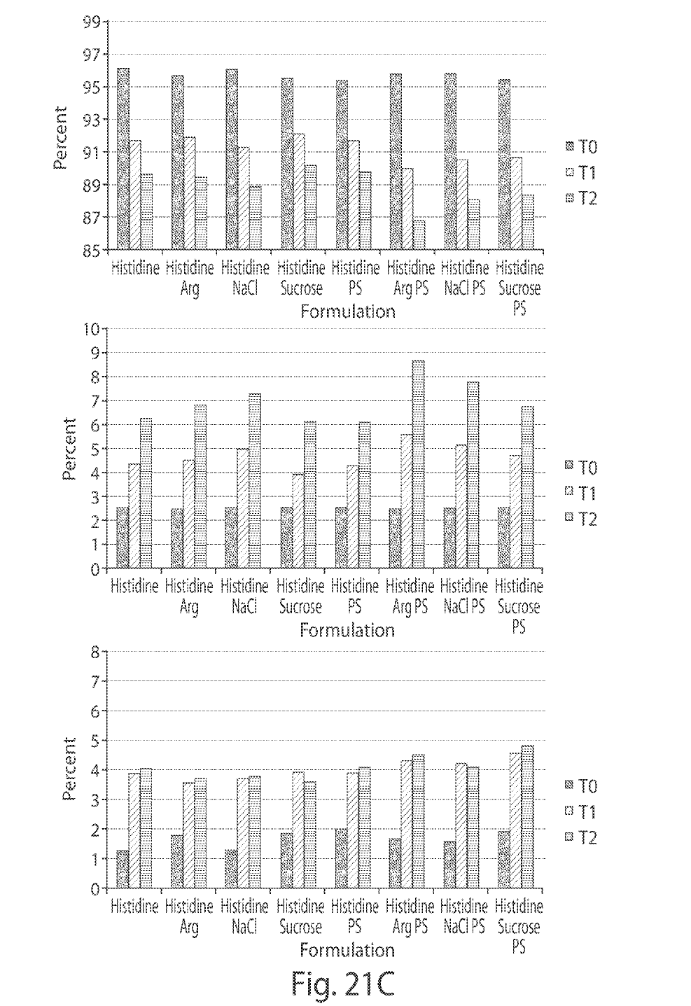

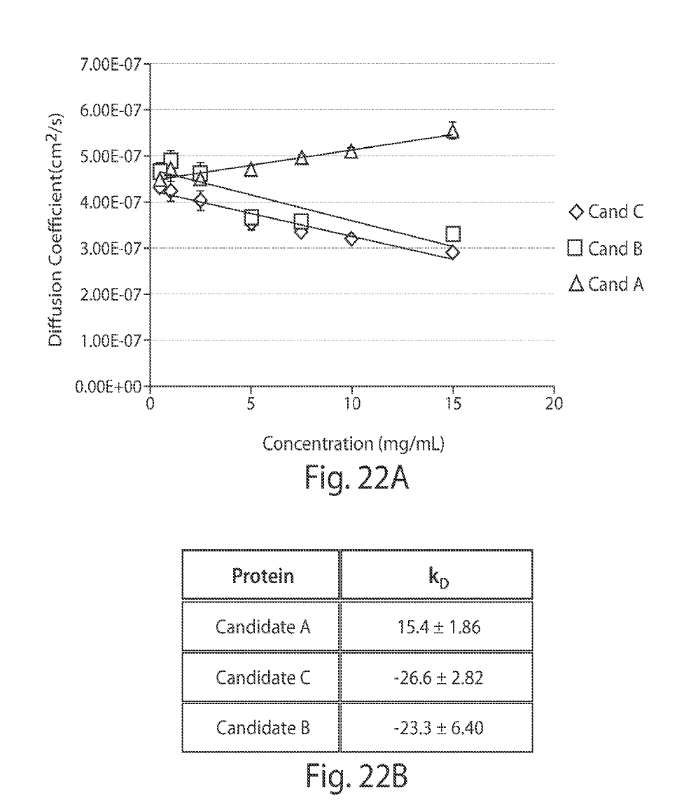

At pH 6, solubility screening indicated that histidine is the favored buffer in this system (FIGS. 20A-20C). All three candidate proteins underwent an excipient screen using SEC (FIGS. 21A-21C). Furthermore, the interaction (k_{D}) as determined by dynamic light scattering, of all proteins measured in 20 mM histidine at a pH 6, showed repulsive interactions between Candidate A and the solute, while Candidates B and C showed attractive interactions with the solute (FIGS. 22A-22B).

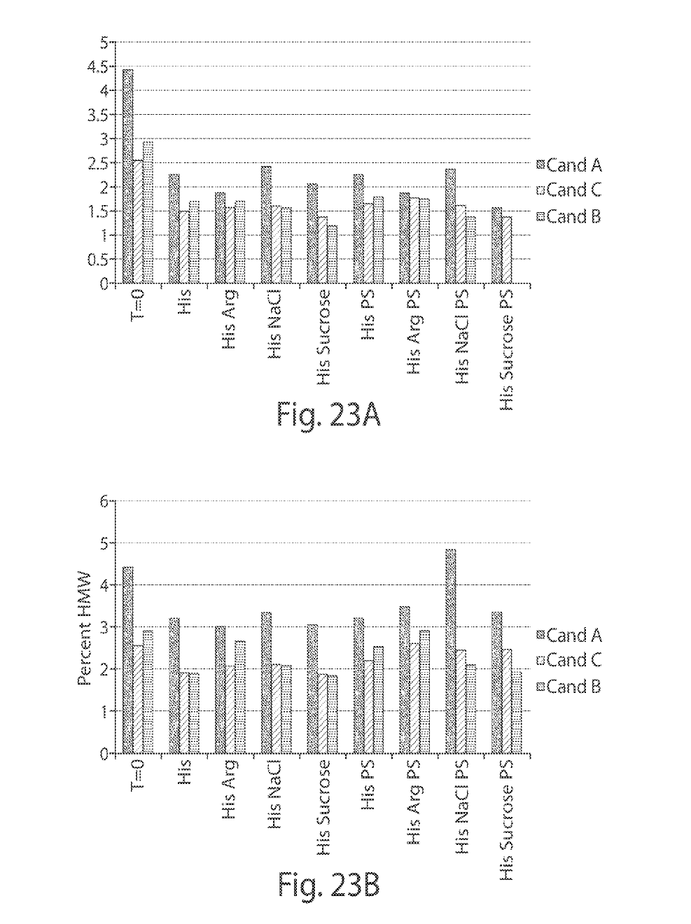

Aggregation of candidate proteins upon agitation and freeze-thaw did not show a significant effect on the studied excipients (FIGS. 23A-23B).

### Example 6

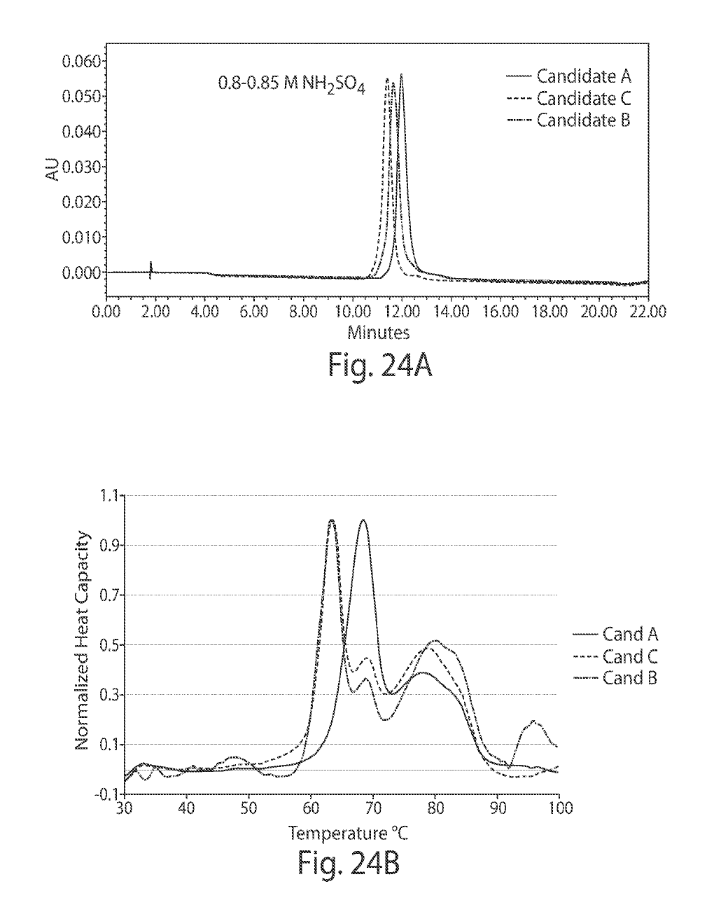

In this example, the initial biophysical screening of three candidates is positive. All three possess reasonable hydrophobicity (FIG. 24A) and thermal stability (FIG. 24B). Candidate A has a positive K_{D}, which suggests good colloidal stability (FIG. 22A).

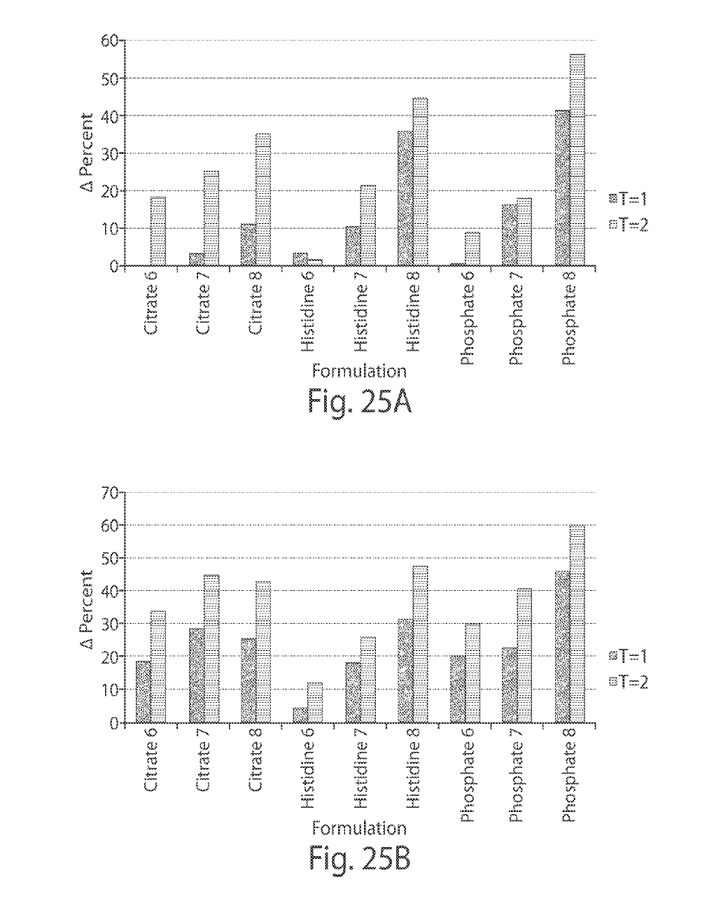

Candidate proteins were formulated in pH 3-8 citrate buffer at 1 mg/mL and incubated at 40° C. for two weeks. All candidates were stable between pH 5 and 8. Two additional buffers were tested at 1 mg/mL; histidine appeared to be superior. Furthermore, the formation of acidic species was the major route of degradation (FIGS. 25A-25B).

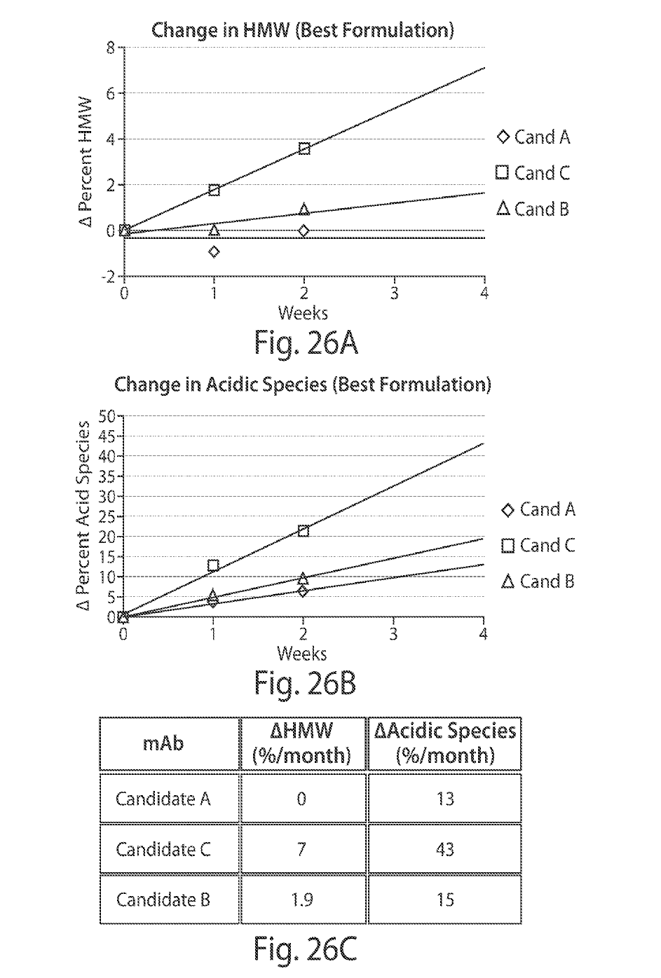

High concentration (100 mg/mL) excipient screening was also performed. Formations included 150 mM NaCl, 150 mM Arg, 5% sucrose, with or without polysorbate 80. Ideally, a candidate should have less than a 3% change in HMW and 15% change in charged isoforms over the four week incubation at 40° C. The results (FIGS. 26A-26C), show that Candidate C rapidly aggregates at high concentrations.

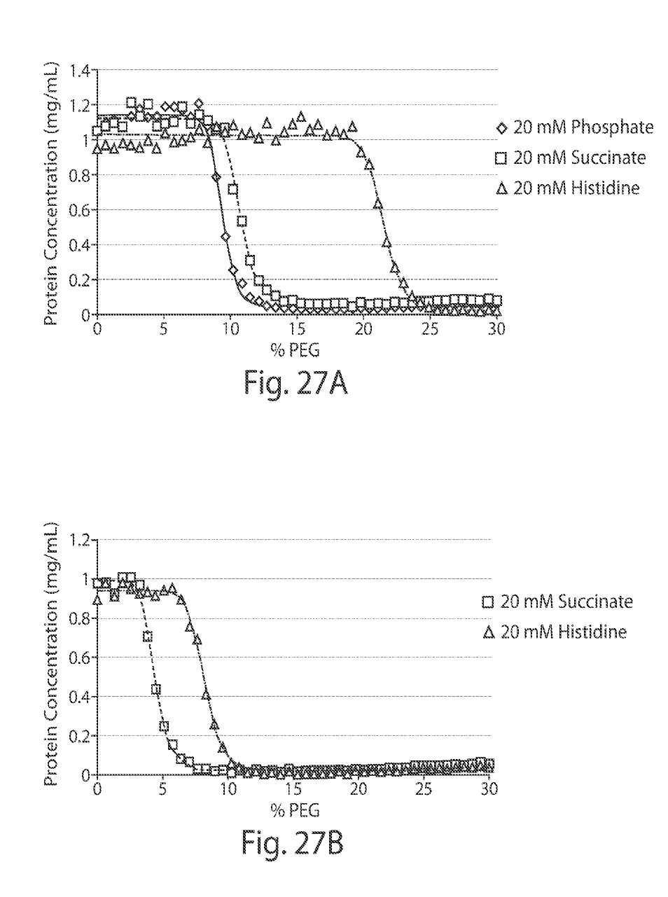

Solubility was assessed by PEG precipitation. The lower concentration formulation (1 mg/mL) was used, as PEG precipitation at low protein concentrations can predict high concentration behavior. The histidine buffer increases the apparent solubility of Candidates A and C. PEG precipitation suggests that Candidate A is stable at a high concentration (>150 mg/mL), and Candidate C has the lowest apparent solubility (114 mg/mL) (FIGS. 27A-27B). Candidate B precipitated during dialysis into the phosphate buffer at 10 mg/mL and therefore could not be analyzed.

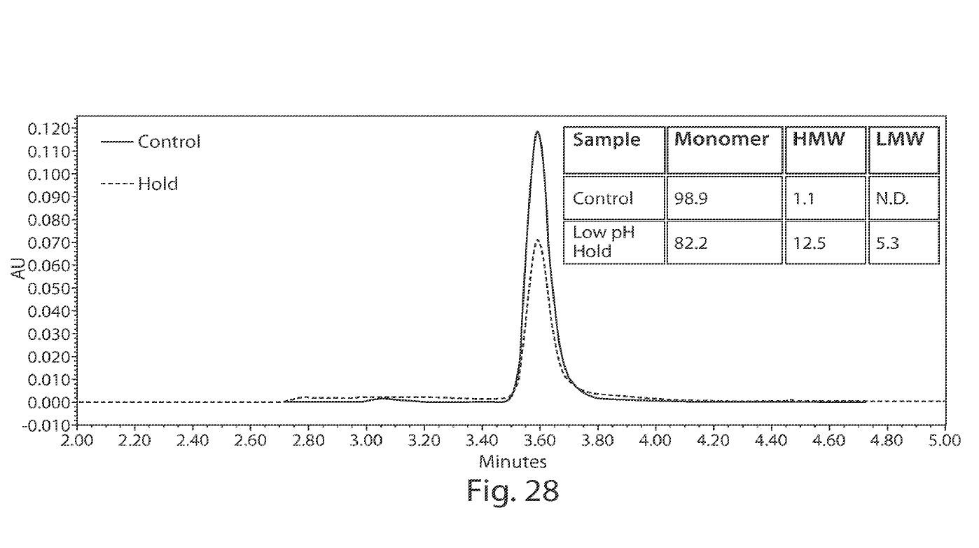

To test low pH hold, 150 mg/mL of Candidate C was diluted to 1 mg/mL in 100 mM glycine to a pH of 3. The sample was held at low pH for five hours and neutralized by the addition of 0.9M bis-tris, 0.24M tris base to a final pH of 7.8. The sample showed lower monomer values, and higher HMW and LMW values than the control (FIG. 28).

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A method of assisting in the selection of a candidate therapeutic protein for pharmaceutical formulation, the method comprising:
a) evaluating a pH effect on one or more properties of a plurality of candidate therapeutic proteins;
b) evaluating a buffer effect on one or more properties of the plurality of candidate therapeutic proteins;
c) evaluating an excipient effect on one or more properties of the plurality of candidate therapeutic proteins; and,
determining a relative stability of each of the plurality of candidate therapeutic proteins based on the pH, buffer, and excipient effects of a)-c), wherein candidate therapeutic proteins that have relatively higher stability are more suitable for pharmaceutical formulation.

2. A method of selecting a pharmaceutical formulation for a candidate therapeutic protein, the method comprising:
a) evaluating a pH effect on one or more properties of a candidate therapeutic protein and determining a pH range within which the candidate therapeutic protein is relatively stable and/or soluble;
b) evaluating a buffer effect on the candidate therapeutic protein within the pH range of a) and determining a relative stability and/or solubility of the candidate therapeutic protein in two or more different buffers;
c) evaluating an excipient effect on one or more properties of the candidate therapeutic protein within the pH range of a) and in a buffer of b) in which the candidate therapeutic protein is relatively stable and/or soluble; and,
determining a formulation for the candidate therapeutic protein that is within the pH range of a), comprises the buffer of b), and comprises the excipient of c).

3. The method of claim 2, wherein the formulation comprises the candidate therapeutic protein at high concentration.

4. The method of claim 2, wherein the stability of the candidate therapeutic protein is evaluated by determining conformational stability, charge, charge isoforms, integrity, and/or chemical modification of the candidate therapeutic protein under conditions a), b), and/or c).

5. The method of claim 2, wherein the solubility of the candidate therapeutic protein is evaluated by determining aggregation of the candidate therapeutic protein under conditions a), b), and/or c).

6. The method of claim 2, wherein the candidate therapeutic protein is selected by comparing stability and/or solubility of the candidate therapeutic protein to a reference protein and/or one or more alternative candidate therapeutic proteins.

7. The method of claim 2, wherein the formulation is selected by comparing protein stability and/or solubility in the formulation to a) a reference protein stability and/or solubility and/or b) protein stability and/or solubility in one or more alternative formulations.

8. The method of claim 2, wherein the pH effect is evaluated by determining one or more stability and/or solubility properties of the candidate therapeutic protein under a plurality of pH conditions between pH 4.0 and pH 8.0; and/or
wherein the buffer effect is evaluated by determining one or more stability and/or solubility properties of the candidate therapeutic protein in a plurality of different buffers; and/or
wherein the excipient effect is evaluated by determining one or more stability and/or solubility properties of the candidate therapeutic protein in the presence of a plurality of different excipients.

9-10. (canceled)

11. A method of developing a biological therapeutic product comprising:
a) evaluating pH effect on one or more properties of at least one biological therapeutic agent and identifying an acceptable pH range within which the at least one biological therapeutic agent is stable;
b) evaluating effect of two or more buffers on one or more properties of the at least one biological therapeutic agent within the acceptable pH range and selecting an acceptable buffer within which the at least one biological therapeutic agent is stable; and
c) evaluating effect of two or more excipients on one or more properties of the at least one biological therapeutic agent within the acceptable pH range in the acceptable buffer and selecting one or more excipients upon which the at least one biological therapeutic agent is stable.

12. The method of claim 11, wherein evaluation of the pH effect in step (a) comprises:
(i) preparing a first batch of multiple solutions each comprising the at least one biological therapeutic agent at a pH from about 1.0 to 13.0 and initially evaluating the one or more properties, optionally wherein the concentrations of the at least one biological therapeutic agent in the first batch solutions are about 1 mg/mL;
(ii) incubating the first batch solutions for a first period of time and firstly evaluating the one or more properties; and
(iii) optionally further incubating the incubated first batch solutions for a second period of time and evaluating the one or more properties.

13. The method of claim 12, wherein evaluation of the buffer effect in step (b) comprises:
(iv) preparing a second batch of multiple solutions with two or more buffers, wherein each solution comprises the biological therapeutic agent and a buffer at a pH within the acceptable pH range, optionally wherein the concentration of the biological therapeutic agent in each solution of the second batch is about 1 mg/mL;
(v) initially evaluating the one or more properties of the biological therapeutic agent in each solution;
(vi) incubating the solutions of the second batch for a third period of time and evaluating the one or more properties of the biological therapeutic agent in each solution; and
(vii) further optionally incubating the solutions of the second batch for a fourth period of time and evaluating the one or more properties of the biological therapeutic agent in each solution.

14. The method of claim 13, wherein evaluation of the excipient effect in step (c) comprises:
(viii) preparing a third batch of multiple solutions each comprising the biological therapeutic agent, the optimal buffer, and one or more excipients at the acceptable pH, optionally wherein the concentration of the biological therapeutic agent in each solution of the third batch is about 50 mg/mL or about 1 mg/mL when subjecting to agitation stress;
(ix) initially evaluating the one or more properties of the biological therapeutic agent in each solution;
(x) incubating the solutions of the third batch for a fifth period of time and evaluating the one or more properties of the biological therapeutic agent in each solution; and
(xi) optionally further incubating the solutions for a sixth period of time and evaluating the one or more properties of the biological therapeutic agent in each solution.

15. The method of claim 14, further comprising subjecting the solutions of the third batch to agitation stress for a fifth period of time and evaluating the one or more properties, optionally further comprising subjecting the solutions of the third batch after agitation stress for the fifth period of time to agitation stress for a sixth period of time, and evaluating the one or more properties.

16. (canceled)

17. The method of claim 14, further comprising subjecting the solutions of the third batch to freeze-thaw stress for a fifth period of time and evaluating the one or more properties, optionally further comprising subjecting the solutions of the third batch after freeze-thaw stress for the fifth period of time to agitation stress for a sixth period of time, and evaluating the one or more properties.

18. (canceled)

19. The method of claim 11, wherein the one or more properties are aggregation and degradation properties, optionally wherein the degradation properties are conformation stability, charge stability, and integrity.

20. (canceled)

21. The method of claim 11, wherein the one or more properties are measured by differential scanning calorimetry (DSC), size exclusion chromatography (SEC), gel electrophoresis (GXII), and/or charge variant analysis (icIEF); and/or
wherein the two or more excipients are Generally Recognized as Safe (GRAS) excipients, optionally wherein the two or more excipients are selected from the group consisting of sugars, polyols, amino acids, and polymers; and/or
wherein the pH in step (a) is from about 1.0 to 12.0.

22-28. (canceled)

29. The method of claim 12, wherein the first period of time is about a week; and/or
wherein the second period of time is about a week.

30. (canceled)

31. The method of claim 11, wherein the method is to formulate the at least one biological therapeutic agent; and/or
wherein the method is to screen the at least one biological therapeutic agent for a biological therapeutic product candidate.

32. The method of claim 14, wherein the method is to screen the at least one biological therapeutic agent for a biological therapeutic product candidate, and wherein the biological therapeutic product candidate has one or more acceptable properties in the second and/or third batch solutions, optionally wherein the acceptable properties are acceptable aggregation and degradation properties.

33-34. (canceled)

35. The method of claim 11, wherein the at least one biological therapeutic agent is a protein, optionally wherein the protein is an antibody.

36. (canceled)
